(19) United States
(12) Patent Application Publication
(10) Pub. No.: US 2014/0005644 A1
(43) Pub. Date: Jan. 2, 2014

(54) DEVICES AND METHODS FOR DERMATOLOGICAL TREATMENT USING FRACTIONAL LASER TECHNOLOGY

(71) Applicant: Alma Lasers Ltd., Caesarea (IL)

(72) Inventors: Ziv KARNI, Kfar Shmaryahu (IL); Nadav BAYER, Zichron Yaakov (IL); Yoav AVNI, Ramat Hasharon (IL)

(21) Appl. No.: 13/923,400

(22) Filed: Jun. 21, 2013

Related U.S. Application Data
(63) Continuation of application No. PCT/IB2011/056017, filed on Dec. 29, 2011.
(60) Provisional application No. 61/429,153, filed on Jan. 2, 2011.

(30) Foreign Application Priority Data
Dec. 31, 2010 (GB) 1022131.5

(51) Int. Cl. A61B 18/20

(52) U.S. Cl. CPC A61B 18/20
USPC 606/9

(57) ABSTRACT

Disclosed are methods for dermatological treatment of an area of skin of a subject that, in some embodiments, comprise moving a light director aperture in a prescribed direction relative to a skin surface and triggering a light source to produce a short-duration pulse of light and directing the produced short-duration pulse of light at a skin surface as a plurality of individual light beams every time the light director aperture travels a prescribed distance relative to the skin surface.

Also disclosed are devices useful for fractional laser therapy to affect dermatological treatment that in some embodiments comprise a trigger functionally associated with a distance measurer functionally associated with a contact surface, the trigger configured to trigger a functionally-associated light source when a distance traveled by the contact surface along a skin surface is a prescribed distance.

### RELATED APPLICATION

The present application is a continuation of PCT/IB2011/056017 filed on 29 Dec. 2011, which gains priority from UK Patent Application 1022131 filed 31 Dec. 2010 and from U.S. Provisional Patent Application No. 61/429,153 filed 2 Jan. 2011. PCT/IB2011/056017 filed on 29 Dec. 2011 is incorporated herein by reference in its entirety.

### FIELD AND BACKGROUND OF THE INVENTION

The invention, in some embodiments, relates to the field of dermatological treatment, and more particularly, but not exclusively, to methods and devices for dermatological treatment using lasers.

The skin is a complex multi-layered organ. The outer layer of the skin is the epidermis having a thickness of between 0.05 mm (eyelids) and 1.5 mm (palms and soles) made up of keratinocytes, melanocytes, Langerhans cells, and Merkel cells in five layers. Under the epidermis is the dermis having a thickness of 0.3 mm (eyelids) to 3 mm (back), primarily comprising collagen, elastic fibers and extrafibrillar matrix in two layers, the upper papillary layer and the lower reticular layer. Under the dermis is the hypodermis housing large blood vessels and nerves primarily comprising fibroblasts, adipose cells, and macrophages.

With time and exposure to various environmental factors such as smoke, sunlight and physical damage the skin acquires an aged appearance, coarse, saggy and including blemishes such as acne scars, wrinkles, fine lines, age spots, uneven skin tone, pigmentation, large pores and the like.

It is often desired to rejuvenate the skin, for medical or for cosmetic reasons. In the art, it is known to rejuvenate the skin by damaging the skin and letting the skin heal. In the thus-rejuvenated skin, pigmentation is more even and the severity of blemishes is reduced. Due to the stimulation of collagen growth and new blood vessels in the dermis, the skin appears smoother and taut.

Methods for damaging the skin to remove blemishes include peeling with abrasives (e.g., ground cherry or olive pits), peeling with chemical solutions (e.g., including phenol, trichloroacetic acid or alpha hydroxy acids) or area ablation by heating (e.g., with electromagnetic radiation such as from a flash bulb).

It has been found that in some instances improved results are achieved when, instead of damaging a large contiguous area of skin, many small areas of the skin are damaged and allowed to heal, a technique known as fractional technology. It is believed that the body can more easily heal a small damaged area surrounded by healthy tissue then a large damaged area. Further, as in fractional technology the area of the damaged skin is relatively small, it becomes possible to damage the area of skin more deeply, leading to more effective treatments of blemishes as well as a greater skin-rejuvenating effect, attributed to more effective stimulation of new collagen growth. In some instances, a fractional technology treatment must be repeated a number of times in the same area of skin to achieve maximal to desired effect.

One typical implementation of fractional technology is fractional laser therapy (e.g., using the Pixel® CO2 from Alma Lasers Ltd., Caesaria, Israel) where a short-duration laser beam is directed at an area of skin to produce a large number of small spots (e.g., 49 spots/cm² or 81 spots/cm², each spot about 0.2 mm diameter). Such treatment has been shown to be effective in stimulating new collagen and blood vessel development, improving skin texture and tone as well as removing fine lines, wrinkles, acne scars and uneven pigmentation. Similar devices have been described, for example, in PCT Patent Publication WO 2008/083305.

It has been found that fractional laser therapy provides better skin rejuvenation compared to area ablation methods. Apparently, while the fluence (total units energy deposited per unit area) of area ablation methods must be limited to avoid permanent scarring, the fluence in fractional laser therapy can be much higher, allowing deeper and more significant damage in each small spot without scarring.

A challenge in implementing fractional laser therapy is to produce the required number and density of spots over a large area of skin.

It is known to divide a single laser beam into a plurality of individual beams that are all simultaneously directed at a skin surface so that each individual beam produces a spot. For example, a device including a single laser light source is functionally-associated with an offset-frame and with a beam splitter that divides (typically using a diffraction beam splitter) a main laser beam produced by the laser light source into a plurality of individual beams with the required density. For use, the offset-frame is placed against the skin so that the laser light source is at a correct distance from an area of skin. The laser light source is fired producing a main laser beam that passes through the beam splitter to be divided into the plurality of individual beams, each individual beam making a spot on the area of skin, so that the desired density of spots is produced on the area of skin. After each firing, a user moves the device to a different area of skin with reference to the offset-frame, generally an area of skin adjacent to the first area of skin, and repeats the process until the whole surface of the skin to be treated is treated in a process analogous to stamping.

A limitation of such beam-splitting methods relates to laser power. For effective treatment, the main laser beam must be sufficiently powerful so that when divided (e.g., into 49 or 81 individual beams), each individual beam has sufficient power to deposit sufficient energy in the skin for effective treatment. In practical terms, bulky, expensive and dangerous lasers are required to produce even the 49 individual beams required to cover an area of only 1 cm². Generally, a pulse width is relatively long, typically between 20 milliseconds and 100 milliseconds.

As a typical treatment requires irradiation of hundreds of cm² of skin, the implementation of such methods using stamping is undesirably time-consuming and difficult. An additional disadvantage of stamping is the danger of overlap between two adjacent areas, that may lead to overexposure, or a gap between two adjacent areas, leading to insufficient treatment.

Further, a given beam splitter produces a fixed pattern of beams, so that spot density cannot be easily changed.

In a variant of devices for implementing beam splitting methods, a single beam is split into a plurality of beams with the help of a 2-dimensional array of microlenses. Such microlens arrays are prohibitively expensive. The individual microlenses have a short focal length so that device must contact the skin for use, a factor that makes treatment of large areas of skin even more difficult. Vaporized waste materials condense and accumulate on the microlenses.

It is also known to direct a single laser producing a single laser beam that is directed with the help of moving mirrors to produce a single spot with any one firing, a method known as “2D scanning”. For example, a device including a single laser light source is functionally-associated with an offset-frame and with a mirror array. For use, the offset-frame is placed against the skin so that the laser light source is at a correct distance from an area of skin. The laser light source is set to repeatedly fire a number of times at a known rate. Between two laser firings, the mirror array is moved under computer control to direct the following laser beam towards the skin to produce a spot at a desired location. After a series of laser firings produces a sufficient number of spots in a first area (usually in a 2-dimensional array pattern), a user moves the device to a different area of skin with reference to the offset-frame, generally an area of skin adjacent to the first area of skin and repeats the process, until the whole surface of the skin to be treated is treated in a process analogous to stamping.

Such methods allow the use of lasers that are modest in price and power, and also allow varying of the pattern and density of the spots. The pulse width is typically short, in the order of 1 to 2 milliseconds. However, the required computer control and accurate mechanically controlled mirror array system that such devices are sensitive, complex and expensive. To ensure the proper geometry of spot (e.g., angle relative to skin surface), the size of the area of skin perforated from a single location is about 1-2 cm². As a typical treatment requires irradiation of hundreds of cm² of skin, the implementation of such methods is undesirably time-consuming and difficult.

It is important to note that the two methods damage the skin through two different mechanisms although superficially both produce an array of spots on the skin.

In beam-splitting methods, characterized by multiple (greater than 40) simultaneous individual beams each of relatively low intensity (1-2 mjoule) for a relatively long duration (greater than 20 milliseconds, typically between 50 and 100 milliseconds) damage is primarily thermal damage caused by increased skin temperature propagating from the impact point through the skin by thermal conduction with relatively little tissue ablation.

In scanning methods, characterized by a single beam of high intensity (100 mjoule) for a short duration (1 to 2 milliseconds) damage is primarily ablation damage caused by localized vaporisation of tissue at the light-impact point with relatively little thermal damage.

Various devices and methods known in the art that potentially provide a background for understanding the teachings herein are described in U.S. Pat. No. 6,171,302; U.S. Pat. No. 6,997,923; U.S. Pat. No. 7,951,138; US patent publications US 2002/120256; US 2007/0093798; US 2009/0131922; EP patent publication EP 1923014A1; and PCT patent publications WO 2001/26573; WO 2009/037641; WO 2008/083305, WO2008/002625 and WO 2005/099369.

### SUMMARY OF THE INVENTION

The invention, in some embodiments, relates to the field of dermatological treatment, and more particularly, but not exclusively, to methods and devices for dermatological treatment.

According to a feature of some embodiments of the invention, there is provided a dermatological treatment device comprising:
a) a trigger functionally associated with a distance measurer functionally associated with a contact surface,
   the distance measurer configured to determine a distance traveled by the contact surface along a skin surface in a prescribed direction when in contact with the skin surface,
   the trigger functionally associatable with a light source configured to produce a short-duration pulse of light when triggered, and
   the trigger configured to repeatedly trigger a functionally-associated light source as said contact surface is moved along said skin surface when the distance traveled is a prescribed distance in said prescribed direction; and
b) functionally associatable with a light source associated with the trigger, a light to director including a light director aperture, wherein the light director is configured to direct a produced short-duration pulse of light from the light director aperture at a skin surface as a plurality of individual light beams.

In some embodiments, the device further comprises a light source configured to produce a short-duration pulse of light when triggered, functionally associated with the trigger and with the light director.

According to an aspect of some embodiments of the invention there is also provided a dermatological treatment device comprising:
a) a light source configured to produce a short-duration pulse of light when triggered;
b) functionally associated with the light source, a trigger functionally associated with a distance measurer functionally associated with a contact surface,
   the distance measurer configured to determine a distance traveled by the contact surface along a skin surface in a prescribed direction when in contact with the skin surface, and
   the trigger configured to repeatedly trigger the light source as said contact surface is moved along said skin surface when the distance traveled is a prescribed distance in said prescribed direction; and
c) functionally associated with the light source, a light director including a light director aperture, wherein the light director is configured to direct a produced pulse of light from the light director aperture at a skin surface as a plurality of individual light beams.

According to an aspect of some embodiments of the invention there is also provided a method of dermatological treatment of an area of skin of a subject, comprising:
a) providing a device comprising a light director with a light director aperture and a trigger-functionally associated with a light source, the light director configured to direct a short-duration pulse of light produced by the light source through the light director aperture at a skin surface when the light source is triggered;
b) moving the light director aperture in a prescribed direction relative to the skin surface; and
c) while the device is moving, every time the light-director aperture travels a prescribed distance relative to the skin surface:
   triggering the light source to produce a short-duration pulse of light, and
   directing the produced short-duration pulse of light at an area of the skin surface as a plurality of individual light beams
      thereby causing a beneficial effect as a result of the pulse of light impinging on the skin surface, wherein the trigger is functionally associated with a contact surface, the moving is while maintaining contact of the contact surface with the skin surface; and the prescribed distance is a distance travelled by the contact surface along the skin surface in the prescribed direction.

According to an aspect of some embodiments of the invention, there is also provided a dermatological treatment device comprising:
a light source configured to produce a short-duration pulse of light when triggered mounted on a device body;
the light source functionally associated with a light director including a light director aperture, wherein the light director is configured to direct a produced pulse of light from the light director aperture as a plurality of individual light beams, so that the pulse of light is focused at a plurality of illuminated points that are spaced from one another along a line lying in focal plane at a predetermined distance from the light director aperture; and
functionally associated with the device body, a distance measurer configured to trigger the light source each time the device body is displaced along the focal plane by a prescribed distance in a prescribed direction transverse to the line of the illuminated points;
   wherein the distance measurer includes:
a measuring wheel mounted on the device body for rotation about an axis generally parallel to the line of points, and
a detector functionally associated with the measuring wheel for generating a triggering signal for triggering the light source in response to each detection of a rotation of the wheel through a preset angle,
   wherein the measuring wheel is operative to make frictional contact with the focal plane; and wherein when the device body is oriented so that the light beams impinge on the focal plane at a preferred angle, the measuring wheel is operative to make frictional contact with the focal plane at a point lying on substantially the same line as, and laterally offset from, the illuminated points.

Unless otherwise defined, all technical and scientific terms used herein have the same meaning as commonly understood by one of ordinary skill in the art to which the invention pertains. In case of conflict, the specification, including definitions, will control.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

As used herein, the indefinite articles “a” and “an” mean “at least one” or “one or more” unless the context clearly dictates otherwise.

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

In the Figures:

FIGS. 1A-1I are views of an embodiment of a device as described herein having a spool-shaped roller;

FIG. 2 is a schematic depiction of a device of FIG. 1 used for implementing a method as described herein;

FIGS. 3A and 3B are schematic depictions of trails of illuminated areas on a skin surface resulting from implementing embodiments of a method as described herein;

FIG. 4 is a view of an embodiment of a device as described herein having a cylindrical roller;

FIG. 5 is a view of an embodiment of a device as described herein devoid of a rolling contact-surface bearing component;

FIGS. 6A-6C are views of an embodiment of a device as described herein; and

FIG. 7 is a view of an embodiment of a device as described herein similar to the device depicted in FIG. 6.

### DESCRIPTION OF SOME EMBODIMENTS OF THE INVENTION

The invention, in some embodiments, relates to the field of dermatological treatment, and more particularly, but not exclusively, to methods and devices for dermatological treatment.

The principles, uses and implementations of the teachings herein may be better understood with reference to the accompanying description and figures. Upon perusal of the description and figures present herein, one skilled in the art is able to implement the invention without undue effort or experimentation. In the figures, like reference numerals refer to like parts throughout.

Before explaining at least one embodiment in detail, it is to be understood that the invention is not necessarily limited in its application to the details of construction and the arrangement of the components and/or methods set forth herein. The invention is capable of other embodiments or of being practiced or carried out in various ways. The phraseology and terminology employed herein are for descriptive purpose and should not be regarded as limiting.

As discussed above, it is known to use fractional laser therapy for the treatment of skin for both cosmetic and medical purposes, where a laser is used to illuminate many small spots on a skin surface, causing localized damage at each spot but leaving the surrounding tissue undamaged.

One method of implementing fractional laser therapy involves splitting a single pulse of laser light into a plurality (typically 49 or 81) of beams distributed to illuminate a like plurality of spots of skin in a 2-dimensional array over a well-defined area, typically 1 cm². Amongst the disadvantages is that the fluence of the single pulse is distributed over many beams, requiring either the use of bulky and expensive very powerful lasers or more moderately powered lasers activated for a relatively long time (pulse widths typically between 20 milliseconds and 100 milliseconds) leading to little ablation damage and much thermal damage at the illuminated spots.

A different method of implementing fractional laser therapy involves directing a single laser beam with the help of moving mirrors to illuminate a single spot at any one time. A plurality of spots is illuminated sequentially to form a 2-dimensional array of illuminated spots over a given area, typically 1 cm². Amongst the disadvantages is the requirement for a complex, expensive and sensitive computer-controlled mirror system. As the laser beam illuminates a single spot at any one time, fluence is very high so that the laser is activated for a short time (pulse widths typically 1 to 2 milliseconds) so there is little thermal damage and much ablation damage.

In both methods, treating a large skin surface is performed by stamping, the laborious relocation and activation of the device, 1 cm² at a time.

According to an aspect of the teachings herein, there is provided a method of dermatological treatment of an area of skin of a subject (a human or a non-human animal), comprising:
a) providing a device comprising a light director with a light director aperture and a trigger functionally associated with a light source, the light director configured to direct a short-duration pulse of light produced by the light source through the light director aperture at a skin surface when the light source is triggered;
b) moving the light director aperture in a prescribed direction relative to the skin surface; and
c) while the device is moving, every time the light-director aperture travels a prescribed distance relative to the skin surface:
   triggering the light source to produce the short-duration pulse of light, and directing the produced short-duration pulse of light at an area of the skin surface as a plurality of individual light beams
      thereby causing a beneficial effect as a result of the pulse of light impinging on the skin surface, wherein the trigger is functionally associated with a contact surface, the moving is while maintaining contact of the contact surface with the skin surface; and the prescribed distance is a distance travelled by the contact surface along the skin surface in the prescribed direction. In some embodiments, the individual light beams impinge on the skin surface substantially simultaneously.

In some embodiments, the light beams are therapeutic light beams, that is to say, are of a wavelength, dimensions, intensity and duration to have a desired therapeutic effect. In some embodiments, the beneficial effect at least partially results from heat damage caused to areas of skin by the light beams. In some embodiments, the beneficial effect at least partially results from ablation of areas of skin by the light beams.

Specifically, as the device is moved in a prescribed direction relative to the skin surface, the light source is repeatedly triggered to produce a pulse of light at prescribed distance intervals. A single produced pulse of light is directed at the skin surface as a plurality of individual light beams, illuminating a plurality of spots of skin, the illumination causing a beneficial effect.

Unlike the art, where a fractional laser device is used by “stamping”, i.e., repeatedly relocated to abutting skin areas and triggered, the teachings herein allow large areas of skin to be treated quickly by moving a suitable device in a prescribed direction so that the device leaves a trail of illuminated spots on a strip of skin. To treat a large surface area, in some embodiments the device is moved in a prescribed direction as described above to treat a first strip of skin, and then relocated and moved in substantially the same way to treat a following strip of skin parallel to the first strip of skin. Additionally, if an operator decides that the intensity of treatment is not sufficient, the method can be repeated for a previously treated area of skin, using the same or a different prescribed direction.

In stamping methods, it is difficult to ensure that any two treated areas exactly abut, so often a significant portion of the skin is left untreated, or a portion of the skin is treated twice, potentially causing overexposure to light and possible damage. Generally, it is simpler to ensure that any two strips of skin treated in accordance with the teachings herein are parallel and abut.

Accordingly, in some preferred embodiments, during the moving of the light director aperture in a prescribed direction relative to the skin surface, the view to where the light beams impinge on the skin surface is unobscured by the device. Such embodiments allow a user to avoid features (and, if desired, carefully maneuver around) on the skin that it is desired to not irradiate (e.g., cuts, wounds, sensitive areas, mucosa). Such embodiments allow a user to view the portions of skin treated (the illuminated spots) to confirm that these are at the desired place on the skin surface and no unexpected or unusual side-effects occur during treatment. Such embodiments make it simpler to ensure that succeeding adjacent strips are separated by a desired distance (not too far apart and not too close together), typically parallel and abutting.

Additionally, in some preferred embodiments, during the moving of the light director aperture in a prescribed direction relative to the skin surface the device does not come in physical contact with where the light beams impinge on the skin surface, helping avoid unwanted irritation or damage to illuminated areas. For example, some such embodiments assist in preventing physical damage caused by scraping by the device and/or dragging of debris into the illuminated areas, two factors that alone or together potentially can lead to infection.

In some embodiments, the device is configured so that the light director aperture is maintained at a substantially constant height from the skin surface during use of the device. In some embodiments, the substantially constant height is at least about 2 cm. In some embodiments, maintaining the light director aperture at a substantially constant height from a skin surface helps assure that a substantially constant effect is maintained at all treated areas of skin, helps prevent obscuring of the view to where the light beams impinge on the skin surface, and helps prevent the device from coming in physical contact with where the light beams impinge on the skin surface.

In some embodiments, the device is configured so that the light director aperture is maintained at a height of not less than 2 cm (in some embodiments not less than 2.5 cm, and in some embodiments not less than 3 cm) from the skin surface during use of the device. In some embodiments, maintaining the light director aperture at such a height helps prevent obscuring of the view to where the light beams impinge on the skin surface, helps prevent the device from coming in physical contact with where the light beams impinge on the skin surface and help avoid contamination of the light-director aperture with debris generated by ablation of the skin. In some such embodiments, the device is configured for directing the plurality of individual light beams to impinge on a skin surface at a preferred angle (in some embodiments, perpendicularly, in some embodiments at an angle of between about 5° and about 60°, or even between about 25° and about 50° from perpendicular to the skin surface) but allows the user to rotate components of the device relative to the skin surface (pitching motion, described in detail below) to change the incident angle and thereby also the height from the skin surface the light director aperture is held. Typically, the device is configured (e.g, by placement of the axis of the rotation to be close to the skin surface during use) so that the typical range of angles does not substantially change the height.

It is important to note that in some embodiments, there is a qualitative difference between known laser fractional technology treatment methods known in the art and the methods disclosed herein.

Specifically, the known methods are static, so that when using a specific commercially available laser light source, the size of a spot (the area of skin illuminated by a single light beam), the number and density of the spots and the duration of illumination (as determined by the laser pulse width) can be selected to achieve a desired therapeutic effect including balancing the amount of thermal damage and the amount of ablation damage. In contrast, in embodiments of the method as described herein, the light director aperture is moving relative to the skin surface. As a result the pulse width is limited to pulse widths that are short enough to avoid substantial negative effects caused by “smearing” of the light spot as the light director aperture is moved. Further, the light source must have a repetition rate that is sufficient to produce a flash of light at the prescribed distance interval. Further, each individual beam of light must be sufficiently intense so that the fluence at the illuminated spot is sufficient to provide a desired effect.

a priori, it is not clear that a method as described herein can achieve the desired beneficial effects considering factors including practical speeds at which the light director aperture is moved relative to a skin surface (especially manually), known light-source repetition rates, known light source power, desired prescribed distances and the linear density and number of light beams to be directed at the skin surface to cause a desired beneficial effect.

The pattern with which the individual light beams impinge on the skin surface is any suitable pattern. That said, in some embodiments the light beams impinge on the skin surface in a one-dimensional array (a line, including a curved line) that in some embodiments is oriented substantially perpendicularly to the movement direction. All things being equal (light-source power, number of individual beams, beam spacing, movement speed) a one-dimensional array allows the illuminated area to be as wide as possible, allowing the greatest surface area to be treated with every light-source pulse.

In some embodiments, the light beams impinge on the skin surface in two one-dimensional arrays (lines), in some embodiments the two one-dimensional arrays are parallel and oriented substantially perpendicularly to the movement direction. In some embodiments the two one-dimensional arrays are staggered. All things being equal (e.g., light-source power, number of individual beams, beam spacing, width of illuminated area, beam cross-sectional area), two one-dimensional arrays allow a laser to operate at a lower repetition rate and/or with a greater movement speed.

The distance between any two spots of a skin surface illuminated by two adjacent light beams is any suitable distance which is generally determined according to clinical factors known in the art of laser fractional technology. That said, in some embodiments, the individual light beams impinge on a skin surface separated by a distance (center to center) of at least about 0.5 mm and even at least about 0.8 mm. In some embodiments, the individual light beams impinge on a skin surface separated by a distance (center to center) of not more than about 2 mm, not more than about 1.5 mm and even not more than about 1.2 mm. In some embodiments, the individual light beams impinge on a skin surface separated by a distance (center to center) of between 0.8 mm and 1.2 mm, for instance 1 mm.

The width (dimension perpendicular to the prescribed direction, for instance length of a one-dimensional array) of the illuminated area of the skin surface is any suitable width, and is determined in conjunction with other parameters as discussed hereinbelow, for example number of light beams, light beam density, light beam fluence, light beam dimensions as well as the shape (especially curvature) of an area of skin to be treated. As is clear to one skilled in the art upon perusal of the disclosure herein, with smaller width (narrower) more parallel strips are needed to treat an area of skin and with greater width (wider) the device is less maneuverable. That said, all things considered, in some embodiments the width of the illuminated area of skin (e.g., of a one-dimensional array) is not less than about 5 mm and even not less than about 10 mm. In some embodiments the width of the illuminated area of skin is not greater than about 30 mm, not greater than about 25 mm and even not greater than about 20 mm. In some embodiments, the width of the illuminated area is between about 10 mm and about 20 mm.

The plurality of individual light beams is any suitable number of light beams greater than 1, preferably greater than 2. The number is generally determined by factors such as the distance between any two beams, the width of the illuminated area and the desired power of each beam. In some embodiments the plurality of light beams is not more than 20 individual light beams, not more than 14 individual light beams and even not more than 10 individual light beams. In some embodiments, there are between 5 and 10 (e.g. 7 or 9) individual light beams, in some embodiments in a 1 cm-wide illuminated area.

As noted above, the light source is triggered to produce a pulse of light every time the light-director aperture travels a prescribed distance in the prescribed direction relative to the skin surface. When the prescribed distance is smaller, illuminated spots on the skin surface from succeeding light source triggerings are closer together while when the prescribed distance is greater, illuminated spots on the skin surface from succeeding light source triggerings are further apart. The prescribed distance can be any suitable prescribed distance. In some embodiments, the prescribed distance is not more than about 5 mm, not more than about 4 mm and in some embodiments not more than about 3 mm. In some embodiments, the prescribed distance is not less than about 0.5 mm and in some embodiments not less than about 0.9 mm. In some embodiments, the prescribed distance is between about 1 mm and about 3 mm.

As the light source is triggered as a function of distance moved, the method is substantially independent of the speed at which the light director aperture is moved. As a result, a user may move the light director aperture as convenient, with no need for maintaining a constant speed and may change the speed, for example, to maneuver the device around anatomical features or even stop moving the device for a short period of time, for example to rest or to reposition the user self for greater comfort, or to examine a portion of the skin surface that has been treated or is to be treated. That said, a speed is (for example, in some embodiments where the light director aperture is moved manually as discussed below) not more than about 10 mm sec⁻¹ and typically up to about 5 mm sec⁻¹.

As noted above, when triggered the light source produces a short-duration pulse of light. Any suitable short-duration pulse of light having any suitable pulse width may be used in implementing the teachings herein. In some embodiments, the pulse width is between about 0.2 milliseconds and about 100 milliseconds. In some embodiments, the pulse width is at least about 0.5 milliseconds. In some embodiments, the pulse width is not more than about 50 milliseconds, and even not more than about 30 milliseconds. That said, the pulse width is preferably short enough so that “smearing” of the illumination spots does not substantially change the efficacy of illumination: such smearing increasing the size of a spot of light and reducing the fluence at each spot. In some embodiments, the pulse width of the pulse of light is not more than about 10 milliseconds, not more than about 5 milliseconds and even not more than about 2.5 milliseconds.

The individual beams of light can have any suitable cross sectional shape although typically the beams have a round cross sectional shape.

The individual beams of light can have any suitable size. In some embodiments, an individual light beam illuminates an area of skin that is not more than about 0.126 mm² (equivalent to a 0.4 mm diameter circle), not more than about 0.0707 mm² (equivalent to a 0.3 mm diameter circle), and even not more than about 0.0314 mm² (equivalent to a 0.25 mm diameter circle). In some embodiments, an individual light beam illuminates an area of skin that is not less than about 0.0020 mm² (equivalent to a 0.05 mm diameter circle), not less than about 0.0050 mm² (equivalent to a 0.08 mm diameter circle), and even not less than about 0.0113 mm² (equivalent to a 0.12 mm diameter circle).

The light source is any suitable light source, for example comprising a pulsed laser or comprising a flash lamp. In some embodiments, the light source is coherent. In some embodiments, the light source is not coherent. In some embodiments, the light source is polychromatic and produces a pulse of light including multiple wavelengths. In some embodiments, the light source is substantially monochromatic. In some embodiments, the light source comprises multiple light emitters (e.g., multiple lasers, light-emitting diodes, flash lamps). In some embodiments, the light source comprises a single light emitter (e.g., a single laser, a single light-emitting diode, a single flash lamp).

That said, in the art of skin treatment it is known that lasers are a preferred source of light for producing a sufficiently long and intense pulse of light having a wavelength that induces a desired effect without substantial negative side effects.

Thus, in some embodiments, the light source comprises a laser (preferably a single laser). Preferably, the laser is an ablative laser emitting light with a wavelength between 2 and 11 microns.

In some embodiments, the light source comprises a CO₂ laser pulsed (by activating and deactivating the laser) to produce pulses of substantially monochromatic light with a wavelength of between 9.6 and 10.6 micrometer, for example 10.6 micrometer. Such lasers are commercially available for skin treatment applications at between 10 W and 100 W. In some embodiments, a 70 W CO₂ laser is preferred for implementing the teachings herein. In some embodiments, the method is implemented so that with each pulse of the light source, each beam deposits between about 10 mjoule and about 200 mjoule, in some embodiments between about 20 and 100 mjoule on a skin surface.

In some embodiments, the light source comprises an Erbium YAG laser producing pulses of substantially monochromatic light with a wavelength of between 2.5 and 3.5 micrometer, for example 3.0 micrometer. Such lasers are commercially available for skin treatment applications at between 100 mjoule to 3000 mjoule. In some such embodiments, the method is implemented so that with each pulse of the light source, each beam deposits between about 1 mjoule and about 300 mjoule, in some embodiments between about 100 and about 250 mjoule on a skin surface.

A suitable light source is a light source having a sufficient firing rate, that is to say, can be repeatedly triggered at a rate sufficient to produce light pulses at a required (not necessarily constant) frequency. It has been found that in some embodiments, the maximal required firing rate is up to about 10 Hz.

As noted above, in some embodiments, the trigger is functionally associated with a contact surface, wherein the moving of the light director aperture is performed while maintaining contact of the contact surface with the skin surface; and wherein the prescribed distance is a distance travelled by the contact surface along the skin surface in the prescribed direction. In some such embodiments, the light director aperture and the contact surface are mutually physically associated through a device body, so that a distance and relative orientation between the light director aperture and the contact surface are fixable during use, so that the pulse of light impinges on the skin surface at a prescribed location and incident angle relative to the contact surface, allowing more accurate and repeatable illumination of areas of skin. This is exceptionally useful when the moving of the light director aperture is performed manually by a person holding the device body.

In some embodiments, the moving of the light director aperture is performed manually by a person holding the device body. Although manual moving of the light director means the speed at which the light director aperture is moved is uncontrolled and varies, it allows the method to be implemented much more simply and efficiently, without the need for complex and prohibitively expensive control systems.

The method described herein may be used for any suitable treatment. In some embodiments, the treatment is a medical treatment. In some embodiments, the treatment is a non-medical, e.g., cosmetic treatment.

In some embodiments, the treatment is an elective non-medical cosmetic treatment. In some embodiments, the treatment is for skin rejuvenation and the beneficial effect is an improved appearance of the area of skin. Such embodiments include treatment of damaged skin, sun-damaged skin, aged skin, wrinkled skin, blemished skin, scarred skin including acne scars and treatment of large skin pores.

The method described herein may be implemented using any suitable device. In some embodiments it is preferred to use a dermatological treatment device as described herein.

According to an aspect of the teachings herein, there is provided a dermatological treatment device comprising:
a) a trigger functionally associated with a distance measurer functionally associated with a contact surface,
   the distance measurer configured to determine a distance traveled by the contact surface along a skin surface in a prescribed direction when in contact with the skin surface,
   the trigger functionally associatable with a light source configured to produce a short-duration pulse of light when triggered, and
   the trigger configured to repeatedly trigger a functionally-associated light source as the contact surface is moved along the skin surface when the distance traveled (as measured by the distance measurer) is a prescribed distance in the prescribed direction; and
b) functionally associatable with a light source associated with the trigger, a light director including a light director aperture configured to direct a produced pulse of light from the light director aperture at a skin surface as a plurality of individual light beams, that in some embodiments impinge on a skin surface substantially simultaneously.

Such devices include, for example, handpieces configured to be reversibly functionally associated with a suitable light source, for example, an existing light source used for fractional laser technology skin treatments such as light sources commercially available from Alma Lasers Ltd. (Caesaria, Israel). In some embodiments, a plurality of such devices are available (e.g., provided separately or as part of a kit) having different characteristics such as number of light beams, light beam size, light beam pattern or length.

In some such embodiments, a device further comprises a light source configured to produce a short-duration pulse of light when triggered, functionally associated with the trigger and with the light director.

According to an aspect of the teachings herein, there is also provided a dermatological treatment device comprising:
a) a light source configured to produce a short-duration pulse of light when triggered;
b) functionally associated with the light source, a trigger functionally associated with a distance measurer functionally associated with a contact surface,
   said distance measurer configured to determine a distance traveled by the contact surface along a skin surface in a prescribed direction when in contact with said skin surface, and
   said trigger configured to repeatedly trigger the light source as the contact surface is moved along the skin surface when the distance traveled (as measured by the distance measurer) is a prescribed distance in said prescribed direction; and
c) functionally associated with the light source, a light director including a light director aperture, wherein said light director is configured to direct a produced pulse of light from the light director aperture at a skin surface as a plurality of individual light beams, that in some embodiments impinge on a skin surface substantially simultaneously.

In some embodiments, the light director is configured so that the light beams impinge on a skin surface in a one-dimensional array (a line, including a curved line), in some embodiments the one-dimensional array is oriented substantially perpendicularly to the prescribed direction.

In some embodiments, the light director is configured so that the light beams impinge on a skin surface in two one-dimensional arrays (lines), in some embodiments the two one-dimensional arrays are parallel and are both oriented substantially perpendicularly to the prescribed direction. In some embodiments, the two one-dimensional arrays are staggered.

In some embodiments, the light director is configured so that the light beams impinge on a skin surface within a skin area having a dimension of not more than about 30 mm, in some embodiments not more than about 25 mm and in some embodiments not more than about 20 mm perpendicular to the prescribed direction. In some embodiments, the light director is configured so that the light beams impinge on a skin surface within a skin area having a dimension of not less than about 5 mm and in some embodiments not less than about 10 mm perpendicular to the prescribed direction.

In some embodiments, the light director is configured so that the light beams impinge on a skin surface separated by a distance (center to center) of at least about 0.5 mm and even at least about 0.8 mm. In some embodiments, the light director is configured so that the light beams impinge on a skin surface separated by a distance (center to center) of not more than about 2 mm, not more than about 1.5 mm and even not more than about 1.2 mm. In some embodiments, the light director is configured so that the light beams impinge on a skin surface separated by a distance (center to center) of between 0.8 mm and 1.2 mm, for instance 1 mm.

In some embodiments, the plurality of individual light beams is not more than 20 individual light beams, not more than 14 individual light beams and even not more than 10 individual light beams. In some embodiments, the light director is configured so that there are between 5 and about 10 (e.g. 7 or 9) individual light beams that impinge on a skin surface, in some embodiments within a skin area having a dimension of about 1 cm perpendicular to the prescribed direction.

In some embodiments, the prescribed distance is not more than about 5 mm, not more than about 4 mm and in some embodiments not more than about 3 mm. In some embodiments, the prescribed distance is not less than about 0.5 mm and in some embodiments not less than about 0.9 mm. In some embodiments, the prescribed distance is between about 1 mm and about 3 mm. In some embodiments, the prescribed distance is fixed. In some embodiments, the prescribed distance is adjustable by a user.

A light source is configured to produce a pulse of light having a suitable pulse width. In some embodiments, the light source is configured to produce a pulse of light having a pulse width of between about 0.2 milliseconds and about 100 milliseconds. In some embodiments, the light source is configured to produce a pulse of light having a pulse width of at least about 0.5 milliseconds. In some embodiments, the light source is configured to produce a pulse of light having a pulse width of not more than about 50 milliseconds, and even not more than about 30 milliseconds.

A light source and/or a beam director of a device are configured so that the individual beams of light have any suitable cross sectional shape although typically the beams of light have a round cross sectional shape.

A light source and/or a beam director of a device are configured so that the individual beams of light have any suitable cross sectional area on a skin surface. In some embodiments, a light source and/or a beam director are configured so that at a skin surface the individual light beams have a cross-sectional area of not more than about 0.126 mm² (equivalent to a 0.4 mm diameter circle), not more than about 0.0707 mm² (equivalent to a 0.3 mm diameter circle), and even not more than about 0.0314 mm² (equivalent to a 0.25 mm diameter circle). In some embodiments, a light source and/or a beam director are configured so that at a skin surface the individual light beams have a cross-sectional area of not less than about 0.0020 mm² (equivalent to a 0.05 mm diameter circle), not less than about 0.0050 mm² (equivalent to a 0.08 mm diameter circle), and even not less than about 0.0113 mm² (equivalent to a 0.12 mm diameter circle).

Any suitable light source may be used in implementing a device as described herein. In some embodiments, a light source comprises a pulsed laser or comprises a flash lamp. In some embodiments, the light source is coherent. In some embodiments, the light source is not coherent. In some embodiments, the light source is polychromatic and produces a pulse of light including multiple wavelengths. In some embodiments, the light source is substantially monochromatic. In some embodiments, the light source comprises multiple light emitters (e.g., multiple lasers, light-emitting diodes, flash lamps). In some embodiments, the light source comprises a single light emitter (e.g., a single laser, a single light-emitting diode, a single flash lamp).

In some embodiments, the light source comprises a laser, preferably a single laser. Preferably, the laser is an ablative laser emitting light with a wavelength between 2 and 11 microns.

In some embodiments, the light source comprises a CO₂ laser configured to be pulsed (for example by repeated activation and deactivation) to produce pulses of substantially monochromatic light with a wavelength of between 9.6 and 10.6 micrometer, for example 10.6 micrometer. Such lasers are commercially available for skin treatment applications at between 10 W and 100 W. In some embodiments, a 70 W CO₂ laser is preferred. In some embodiments, the laser is chosen, together with other device parameters, so that with each pulse of the light source, each beam deposits between about 10 mjoule and about 200 mjoule, in some embodiments between about 20 and 100 mjoule on a skin surface.

In some embodiments, the light source comprises an Erbium YAG laser producing pulses of substantially monochromatic light with a wavelength of between 2.5 and 3.5 micrometer, for example 3.0 micrometer. Such lasers are commercially available for skin treatment applications at between 100 mjoule to 3000 mjoule. In some embodiments, the laser is chosen together with other device parameters, so that with each pulse of the light source, each beam deposits between about 1 mjoule and about 300 mjoule, in some embodiments between about 100 and about 250 mjoule on a skin surface.

A light source is configured to produce pulses of light at a required (not constant) repetition rate, that is to say, is configured to be repeatedly triggered at a rate sufficient to produce light pulses at a required frequency. It has been found that in some embodiments, the maximal required firing rate is up to about 10 Hz.

A light director is a component of a device as described herein configured to direct a pulse of light produced by the light source and from a light director aperture at a skin surface as a plurality of individual light beams.

In some preferred embodiments a light director is a passive optical element substantially devoid of moving parts (e.g., moving mirrors and lenses). Such a passive optical element is robust, cheap, and not sensitive or delicate. In some embodiments, such as when the light source comprises a single light emitter, the light director comprises a microlens array or a beam splitter (e.g., a diffraction beam splitter, such as available from MEMS Optical (Huntsville Ala.)). For example, in some embodiments where the plurality of individual light beams impinges on a skin surface in a 1 dimensional array, a microlens array is a linear microlens array or a beam splitter is a linear diffraction beam splitter. As embodiments of the device generally require only a small number of individual light beams (e.g., 7 light beams in a line compared to 49 light beams as known in the art), a microlens array or beam splitter of a device as described herein has, for example, higher performance for the same price or the same performance for a lower price, compared to a microlens array or beam splitter (respectively) of prior fractional laser therapy devices. Consequently, in some embodiments, a portion of a light director is configured to be user-replaceable, allowing the characteristics of the beams to be changed by the user. For example, in some such embodiments, a light director is provided with a set of replaceable linear diffraction beam splitters, each such beam splitter having different characteristics such as number of individual beams of light, shape of the beams of light and dimensions of the beams of light.

In some embodiments, the individual light beams are convergent, that is to say, each individual light beam emerges from the light director aperture to converge at a focal point. A challenge in some such embodiments is that, since a beam is convergent, the cross sectional area of the light beam at different parts of the skin surface may vary due to varying features on the skin surface (bumps, freckles), the natural curvature of the skin and underlying tissue or even due to very slight variations of the height of the light director aperture from the skin surface or of the incident angle with which the beams impinge on the skin surface during movement of the device.

In some embodiments, the individual light beams are not convergent. By not convergent is intended that a light beam changes (increases or decreases) in cross section by not more than about 10% from the light director aperture to the skin surface. In embodiments where a device is configured having a preferred angle at which the light beams impinge on the skin surface, a not convergent light beam is a light beam that changes (increases or decreases) in cross section by not more than about 10% from the light director aperture to the skin surface when the beams impinge at the preferred angle.

In some embodiments, the device is configured so that the view to where the light beams impinge on the skin surface is unobscured by the device. Such embodiments allow a user to avoid features (and, if desired, carefully maneuver around) on the skin that it is desired to not irradiate (e.g., cuts, wounds, sensitive areas, mucosa). Such embodiments allow a user to view the portions of skin treated (the illuminated spots) to confirm that these are at the desired place on the skin surface and no unexpected or unusual side-effects occur during treatment. Such embodiments make it simpler to ensure that succeeding adjacent parallel strips are separated by a desired distance (not too far apart and not too close together).

In some embodiments, the device is configured so that during use the device does not come in physical contact with where the light beams impinge on the skin surface, that is to say already treated areas of skin. Such embodiments help avoid unwanted irritation or damage to illuminated areas. For example, some such embodiments assist in preventing physical damage caused by scraping by the device and/or dragging of debris into the illuminated areas, two factors that alone or together potentially can lead to infection.

In some embodiments, the device is configured so that the light director aperture is maintained at a substantially constant height from the skin surface. In some embodiments, the substantially constant height is at least about 2 cm. In some embodiments, maintaining the light director aperture at a substantially constant height from a skin surface helps assure that a substantially constant effect is maintained at all treated areas of skin, helps prevent obscuring of the view to where the light beams impinge on the skin surface, and helps prevent the device from coming in physical contact with where the light beams impinge on the skin surface.

In some embodiments, the device is configured so that all of the plurality of individual light beams impinge on a skin surface at substantially the same incident angle. Such a constant incident angle assists in ensuring that spots of damage caused to the skin by neighboring light beams remain separated by healthy tissue.

In some such embodiments, a device is configured so that during use the light director aperture is maintained at a substantially constant angle relative to a skin surface so that the light beams impinge the skin surface at a substantially constant incident angle (from perpendicular) during use. In some embodiments, the incident angle is between about 5° and about 60° from perpendicular to a skin surface. In some embodiments, the incident angle is between about 15° and about 45° from perpendicular to a skin surface. As described above, in some embodiments, the device is configured having a preferred incident angle at which the light beams impinge on the skin surface.

In some embodiments, the device is configured so that the light director aperture is maintained at a height of not less than 2 cm (in some embodiments not less than 2.5 cm, and in some embodiments not less than 3 cm) from the skin surface during use of the device. Typically, such a height is not more than about 15 cm, not more than about 10 cm and even not more than about 7.5 cm. Maintaining the light director aperture at a height above the skin surface allows a view to the treated area that is unobstructed by components of the device, as well as unobstructed by debris and smoke produced during operation. Such a height allows clearing of debris and smoke by suction and/or blowing. Such a height assists in preventing the debris and smoke from obstructing the path of the light beams to the skin surface and prevents accumulation of debris on the light director aperture. In some embodiments, maintaining the light director aperture at such a height helps prevent obscuring of the view to where the light beams impinge on the skin surface, helps prevent the device from coming in physical contact with where the light beams impinge on the skin surface and help avoid contamination of the light-director aperture with debris generated by ablation of the skin. In some such embodiments, the device is configured for directing the plurality of individual light beams to impinge on a skin surface at a preferred angle (in some embodiments, perpendicularly, in some embodiments at an angle of between about 5° and about 60°, or even between about 25° and about 50° from perpendicular to the skin surface) but allows the user to rotate components of the device relative to the skin surface (pitching motion, described in detail hereinbelow) to change the incident angle and thereby also the height from the skin surface the light director aperture is held. Typically, the device is configured (e.g, by placement of the axis of the rotation to be close to the skin surface during use) so that the typical range of angles does not substantially change the height.

In some embodiments, the height is a substantially constant height, In some embodiments each of the individual light beams is divergent or convergent (focused). Consequently, in such embodiments, a constant height assists in ensuring that the spots of light have the same cross sectional area (and consequently same fluence) when impinging on a skin surface area.

In some embodiments, the contact surface of the distance measurer and the light director aperture of the light director are mutually physically associated through a device body, so that a distance between the light director aperture and the contact surface is defined and fixable during use, so that—during use of the device the light director aperture is maintained at a height of at least about 2 cm from a skin surface, in some embodiments a substantially constant height of at least about 2 cm.

In some such embodiments, the distance between the light director aperture and the distance measurer is user adjustable, allowing the height of the light director aperture from a skin surface to be adjusted by a user.

In some embodiments, the contact surface of the distance measurer and the light director aperture of the light director are mutually physically associated through a device body, so that a relative orientation between the light director aperture and the contact surface is defined and fixable, so that during use of the device the light beams impinge on a skin surface at a prescribed incident angle and at a prescribed location relative to the contact surface.

In some such embodiments, the relative orientation between the light director aperture and the distance measurer is user adjustable, allowing the incident angle of the light beams to a skin surface to be adjusted by a user.

In some embodiments, the device is configured to be hand-operable, allowing a user to manually move the contact surface along a skin surface in the prescribed direction. A user can move such a device at any speed as required. That said, a typical “fast speed” is between about 5 cm sec⁻¹ and about 10 cm sec⁻¹, while a very fast speed is not more than about 20 cm sec⁻¹.

As noted above, a device comprises a trigger functionally associated with a distance measurer functionally associated with a contact surface, the distance measurer configured to determine a distance traveled by the contact surface along a skin surface in a prescribed direction when in contact with the skin surface. Any suitable distance measurer may be used in implementing the teachings herein.

In some embodiments, a distance measurer is substantially similar to a computer mouse, typically comprising an optoelectronic sensor functionally associated with an image-processor. The sensor functions as a camera that periodically acquires images while the image processor compares succeeding images and translates changes in the images to distance. Such devices typically include a dedicated distance-measurer illuminator (such as an LED or laser diode). In some embodiments, such a distance measurer is configured (as known in the art of computer mice) to determine whether or not there is contact with a surface.

In some embodiments, the distance measurer comprises a mechanical rolling component with a rolling surface constituting at least a part of the contact surface, the rolling component configured to rotate around an axis that is substantially perpendicular to the prescribed direction when the contact surface contacts a skin surface. In some such embodiments, the distance measurer is very simple and reliable, so that determining a distance moved by the contact surface along a skin surface requires no processor or calculations. Accordingly, in some such embodiments, the device is devoid of a digital processor for determining the distance moved by the contact surface along a skin surface. Instead, the extent of rolling of the rolling component is monitored as a measure of the distance the contact surface has moved. Every time the mechanical rolling component rotates a prescribed amount, corresponding to the prescribed distance the contact surface is moved along the skin surface, the trigger triggers the light source. Such monitoring can be done using any suitable method (such as mechanical methods, gears, wheels, microswitches, electroptical methods, apertures with light sources and the like).

Embodiments comprising a mechanical rolling component including an axis that is substantially perpendicular to the prescribed direction, for example where the distance measurer comprises a shaft encoder have a number of advantages over an optoelectronic sensor. Such mechanical rolling components directly determine a distance traveled and therefore are not dependent on the optical properties of the skin surface. Further, such mechanical rolling components rotate around the axis and therefore measure a distance traveled by the contact surface only in the prescribed direction as opposed to an optoelectronic sensor that potentially measures a distance traveled in any direction.

In some embodiments, a device comprises a component configured to assist in maintaining a desired orientation of the light director aperture relative to a skin surface during use, for example by preventing tilting (listing around the desired direction) of the light director aperture, or for maintaining a desired distance (height) between the light director aperture and a skin surface. In some embodiments, such a component is a component separate from the contact surface.

In some embodiments, at least a portion of the contact surface is configured to assist in maintaining a desired orientation of the light director aperture relative to a skin surface during use, for example by preventing tilting of the light director aperture.

For example, in some such embodiments, the contact surface comprises two (or more) spaced-apart surfaces, especially flanking at least one, preferably all, of the light beams.

For example, in some such embodiments, the contact surface is relatively wide (in a direction perpendicular to the prescribed direction, e.g., at least 1 cm, at least 2 cm wide.

In some embodiments, the contact surface is configured to flatten a skin surface that the plurality of individual light beams impinge on.

In some embodiments, the contact surface is user-replaceable, allowing greater hygiene.

In some embodiments, the contact surface of the distance measurer and the light director aperture of the light director are mutually physically associated through a device body, so that during use of the device, a distance between the light director aperture and the contact surface is fixed, so that the device is configured to allow rotation of the device body in a plane including the prescribed direction and substantially perpendicularly to a skin surface while maintaining the contact surface in contact with a skin surface.

In some embodiments, the contact surface of the distance measurer and the light director aperture of the light director are mutually physically associated through a device body, so that during use a distance between the light director aperture and the contact surface is fixed, the device further comprising a flanking component configured to flank a target area of the skin surface, and the device configured (especially the light director) so that the individual light beams impinge on the target area of the skin surface when impinging on the skin surface at a preferred angle.

In some embodiments, the flanking component comprises two distinct spaced-apart surfaces configured to contact a skin surface at two respective distinct spaced-apart locations during use of the device. In some embodiments, at least a portion of the target area is located between the two spaced-apart locations, in some embodiments when the device is used so that the light beams impinge on the skin surface at a preferred angle. In some embodiments, substantially all of the target area is located between the two spaced-apart locations, especially when the device is used so that the light beams impinge on the skin surface at a preferred angle. In some embodiments, the device is configured so that the individual light beams impinge proximal (in some embodiments within not more than 1 mm, in some embodiments within not more than 0.5 mm) to a line drawn between the two spaced-apart locations on a skin surface when impinging on the skin surface at a preferred angle. In some embodiments, a preferred angle is perpendicular to the skin surface. In some embodiments, a preferred angle is between about 5° and about 60° from perpendicular to the skin surface. In some embodiments, a preferred angle is between about 25° and about 50° from perpendicular to the skin surface.

In some embodiments, the contact surface constitutes a portion of the flanking component. In some embodiments, the distance measurer comprises a mechanical rolling component (such as a wheel) constituting a portion of the flanking component, the mechanical rolling component having a rolling surface constituting at least a part of the contact surface, the rolling component configured to rotate exclusively around an axis that is substantially perpendicular to the prescribed direction when the contact surface contacts a skin surface.

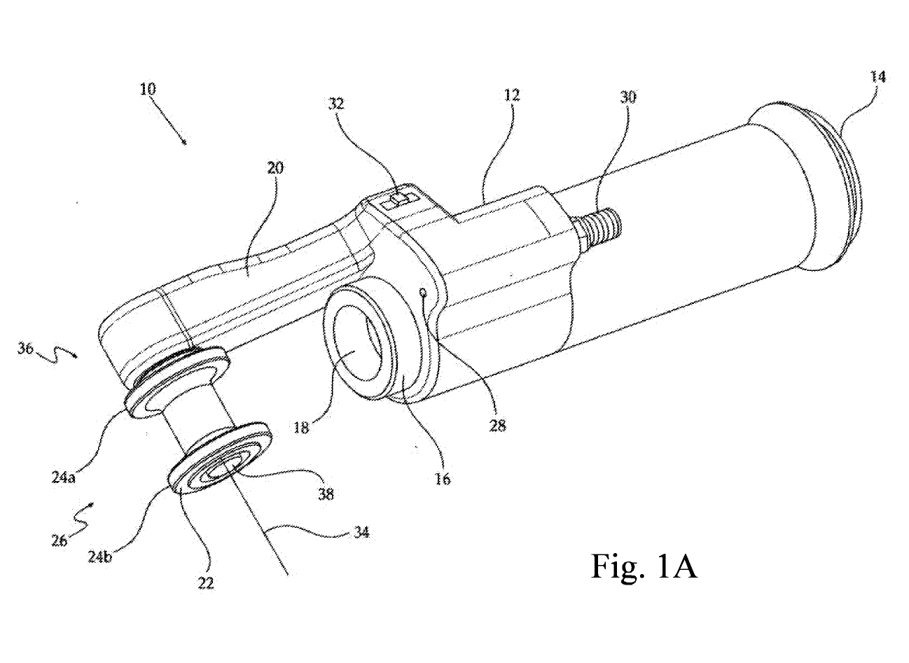

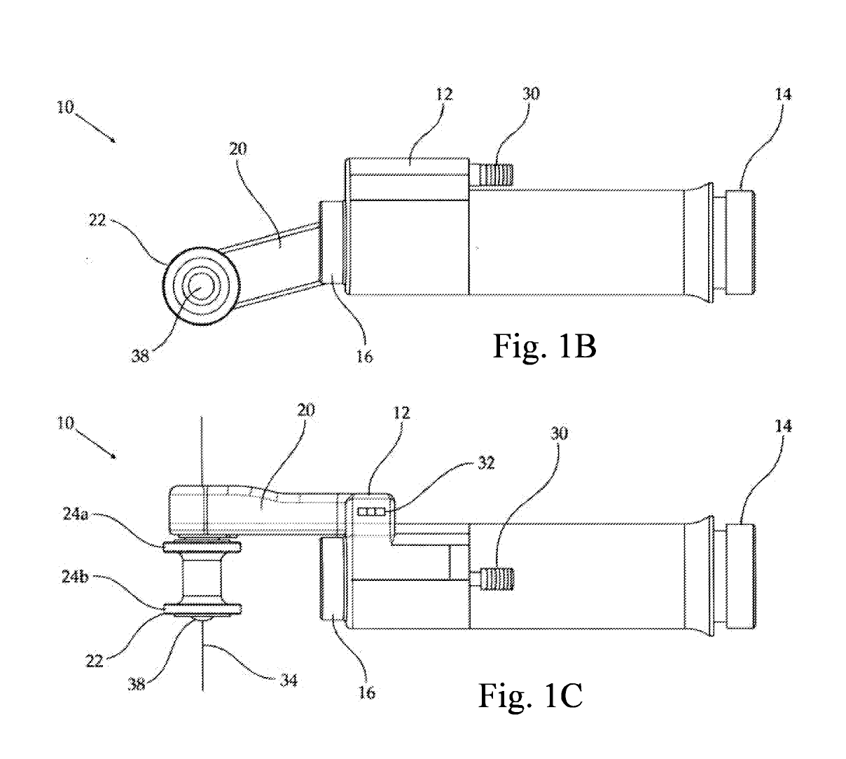

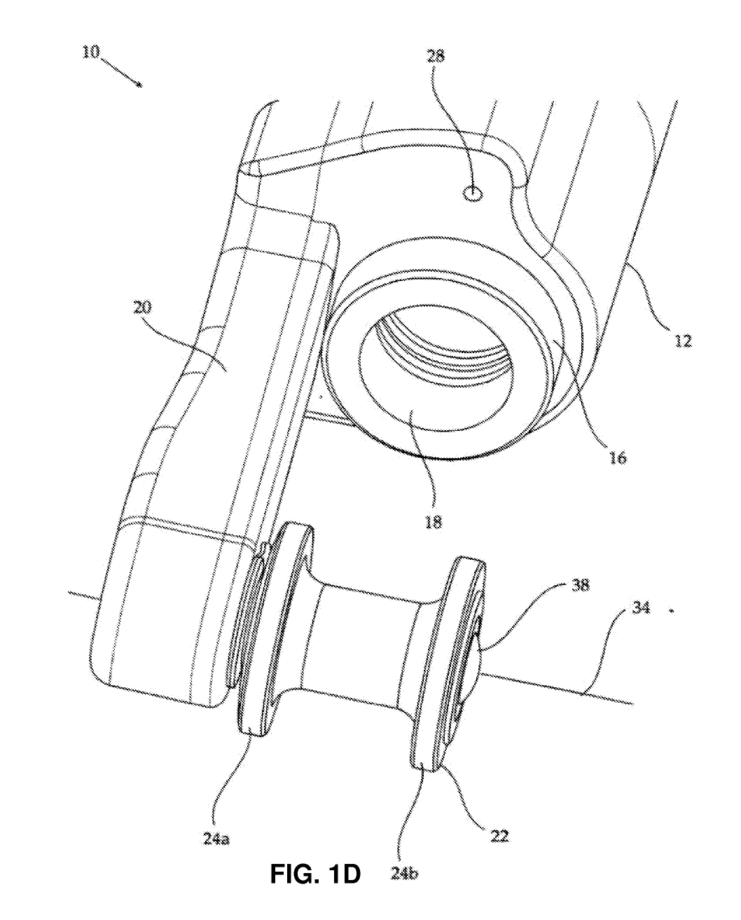

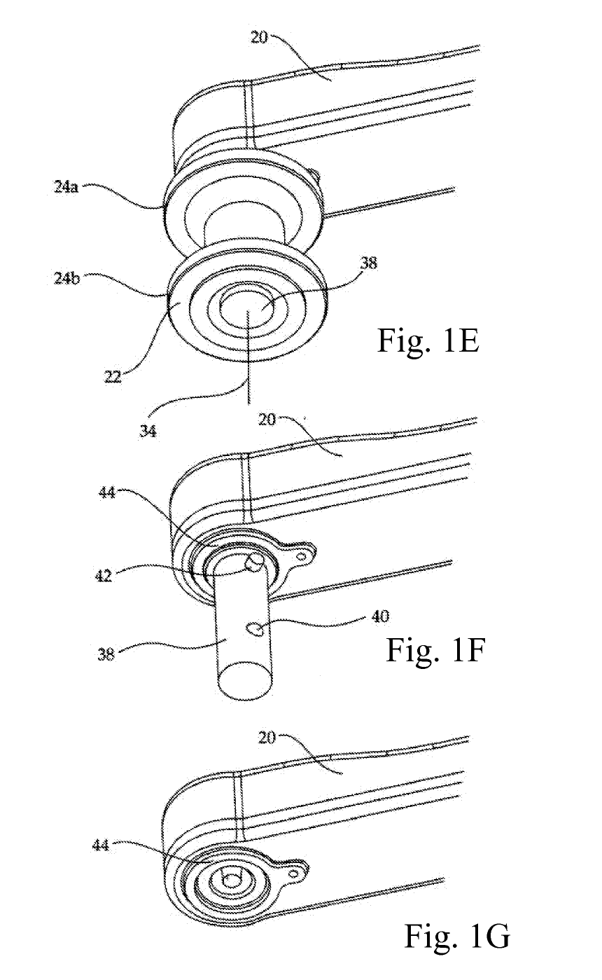

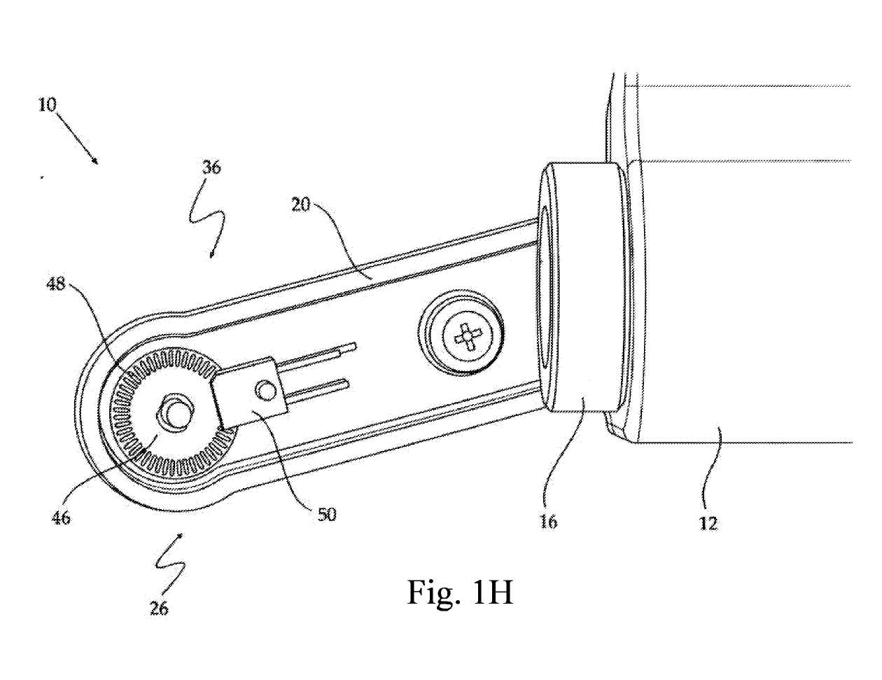

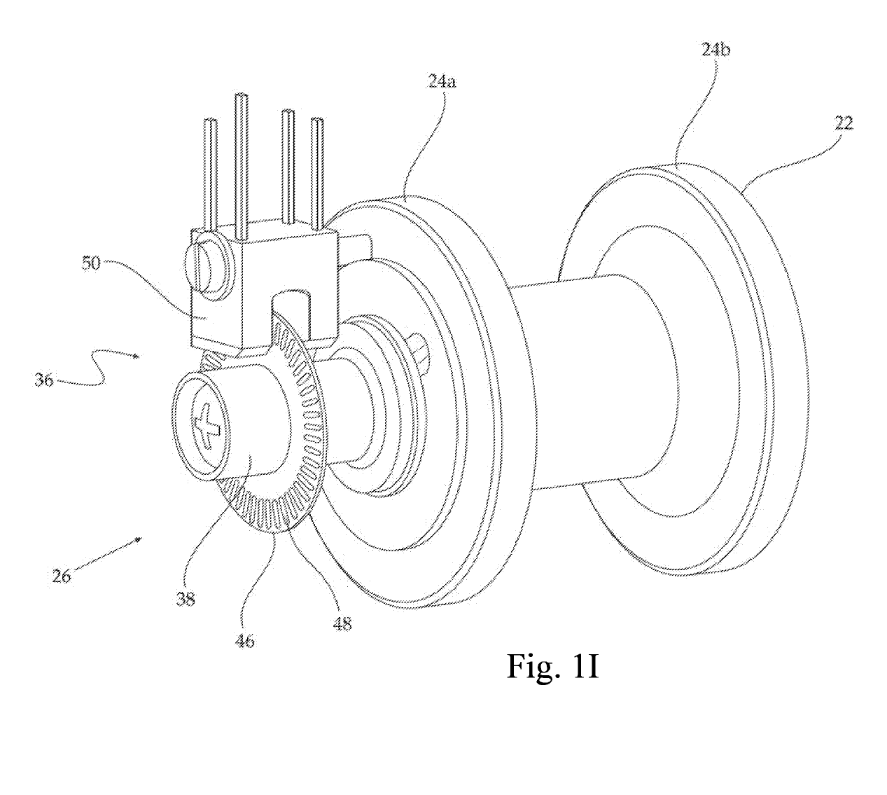

An embodiment of a dermatological treatment device as described herein, a dermatological treatment device 10, is schematically depicted in FIG. 1, in perspective (FIG. 1A), side-view (FIG. 1B), top-view (FIG. 1C), detailed close-up views (FIGS. 1D, 1E, 1F, 1G) and details of a trigger (1H and 1I).

Device 10 comprises a device body 12 containing various components including a light-source connector 14, a user-replaceable light director 16 including a light-director aperture 18, a spacer arm 20 to which distal end is attached a spool-shaped roller 22 bearing a contact surface 24 comprising two spaced-apart parts 24a and 24b, which is functionally associated with a trigger 26, and an air port 28 functionally associated with a nipple connector 30 through an air conduit (not depicted). An additional component of trigger 26 is distance selector switch 32.

As seen in FIG. 1, spool-shaped roller 22 substantially comprises an elongated central member arranged around a rotation axis 34 where at or near both ends of the elongated central member are cylindrical end-members of lesser width and greater diameter, the rims of the end-members constituting parts 24a and 24b of contact surface 24. An advantage of the spool-shape of roller 22 is that the end members of the spool-shape which surfaces constitute contact surface parts 24a and 24b also act to clearly delineate the skin area that is illuminated with the beams of light to a user. Specifically, device 10 is configured so that the beams of light from light director aperture 18 are directed to pass between parts 24a and 24b of contact surface 24 and thus fall inside a skin surface delineated by parts 24a and 24b of contact surface 24.

Device 10 is configured to be hand-operable. Specifically, device body 12 is configured to be easily holdable in a human hand and allows a user to manually move contact surface 24 along a skin surface in a prescribed direction.

Due to the shape and size of device body 12 and of spacer arm 20, the angle of light director aperture 18 relative to a skin surface along which contact surface 24 is moved, and consequently the incident angle of light beams emerging from light director aperture 18, is substantially constant (30°±5° from perpendicular) and the height from the skin surface is substantially constant (about 5 cm) during manual use.

Device 10 is configured as a handpiece that is reversibly functionally associatable with an external light source, for example a cosmetic laser light source from Alma Lasers Ltd (Caesaria, Israel) including a CO₂ or an Erbium YAG laser suitable for cosmetic applications. Specifically, the light-source trigger input and the light-output of the external light source are functionally associated with light director 16 and trigger 26, respectively, by coupling a suitably-configured light-source adaptor to device body 12 through light-source connector 14. Thus, a light-source triggering signal generated by trigger 26 can be received by the light source and a pulse of light produced by the light source in response to the light-source triggering signal passes through device body 12 to light-director 16.

In device 10, light director 16 is a user-replaceable diffraction beam splitter (from MEMS Optical (Huntsville Ala.). Light director 16 is configured so that a pulse of light such as produced by a functionally-associated light source entering device body 12 through light-source connector 14 is directed at a skin surface in proximity of roller 22 between the rims of roller 22 (that constitute the two contact surface parts 24a and 24b) as a 1-cm wide one-dimensional array of seven individual light beams, the one-dimensional array being parallel to an axis 34 and perpendicular to the prescribed direction. At a skin surface, each individual light beam has a round cross sectional shape of 0.03 mm² (a 0.2 mm diameter circle).

Trigger 26 is functionally associated with a distance measurer 36 discussed in detail hereinbelow and various additional components, some housed in spacer arm 20 including a processor (not depicted) and distance selector switch 32.

The processor comprises a suitably configured electronic microprocessor. The processor is configured to be functionally associated with a light source: when a light source is associated with device 10 through light-source connector 14, the processor receives electrical power for operation therefrom. The processor is configured to monitor a signal received from distance measurer 36 indicating a distance traveled by contact surface 24 along a skin surface in the prescribed direction and to send a signal triggering the light source to produce a short-duration pulse of light when the distance traveled corresponds to a prescribed distance as selected by a user through distance selector switch 32.

Distance selector switch 32 is a switch functionally associated with the processor having four states: off (the processor does not function), prescribed distance is 0.59 mm, prescribed distance is 1.18 mm and prescribed distance is 1.77 mm.

As seen in FIGS. 1H and 1I, trigger 26 is functionally associated with a distance measurer 36 functionally associated with a contact surface 24, distance measurer 36 configured to determine a distance traveled by contact surface 24 along a skin surface in a prescribed direction when in contact with the skin surface. Specifically, distance measurer 36 comprises a mechanical rolling component, spool-shaped roller 22 of annealed 316 stainless steel, measuring 2 cm wide (rim to rim) and 18 mm in diameter mounted on an axle 38, where the two spaced-apart parts 24a and 24b of contact surface 24 are the rims of spool-shaped roller 22.

For greater hygiene, roller 22 including contact surface 24 is configured to be user-replaceable. As seen in FIGS. 1E and 1F, roller 22 is reversibly mounted on axle 38. For removal, a user holds spacer arm 20 and pulls roller 22 in parallel to axle 38 (FIG. 1E) so that roller 22 is released from the rest of device 10 to be cleaned or discarded. For attachment, a user threads an axial hole on inner face of roller 22 over axle 38 and forces roller 22 past outwardly-biased spring-loaded retainer pin 40 in axle 38 so that a fixed pin 42 attached to axle 38 enters a slot (not depicted) in the inner face of roller 22. The force applied by retainer pin 40 prevents roller 22 from disconnecting from axle 38 while fixed pin 42 guarantees that any rotation of roller 22 is translated into rotation of axle 38 with substantially no play.

Axle 38 is rotatably mounted near the distal end of spacer arm 20 through bearing 44. Fixedly secured to axle 38 is perforated disk 46 (FIGS. 1H and 1I), a 0.3 mm thick stainless steel disk having 48 perforations 48 arranged near the periphery thereof. Surrounding a portion of an edge of perforated disk 46 is microphotosensor 50 (Omron Corporation, Kyoto, Japan) functionally associated with the processor. Microphotosensor 50 comprises a light-emitting diode and a photosensor and when activated (by receiving power from the processor) outputs one of two signals: a) a signal indicating that light from the light-emitting diode is detected by the photosensor and b) a signal indicating that light from the light-emitting diode is not-detected by the photosensor. The output of microphotosensor 50 is sent to the processor. Accordingly, the distance measurer of device 10 includes a shaft encoder.

Roller 22 is configured to rotate axle 38 and consequently perforated disk 46 around axis 34 in bearing 44. around axis 24 together with axle 38 and perforated disk 46. During a typical use of device 10, contact surface 24 is placed against a skin surface and device 10 moved in a prescribed direction perpendicular to axis 34. Roller 22 rotates to a degree that is related to the distance moved, rotating axle 38 and perforated disk 46. During rolling, the signal sent by microphotosensor 50 to the processor alternates between “detected” and “not-detected”. As the diameter of the rims of roller 22 is 18 mm and there are 96 detected/not detected transitions per full rotation of roller 22, each transition between detected/not detected signal from microphoto sensor 50 corresponds to a traveled distance of 0.59 mm.

As noted above, contact surface 24 comprises two spaced-apart surfaces 24a and 24b flanking all of the light beams from light director aperture 18. In such a way, contact surface 24 is configured to assist in maintaining a desired orientation of light director aperture 18 relative to a skin surface during use: as contact surface 24 is made up of two spaced-apart surfaces 24a and 24b, contact surface 24 assists in preventing tilting (in a plane perpendicular to the prescribed direction) of light director aperture 18 relative to the skin surface.

Not depicted is that trigger 26 is functionally associated with a foot pedal that functions as a safety switch. The processor is configured to function only when the foot pedal is depressed.

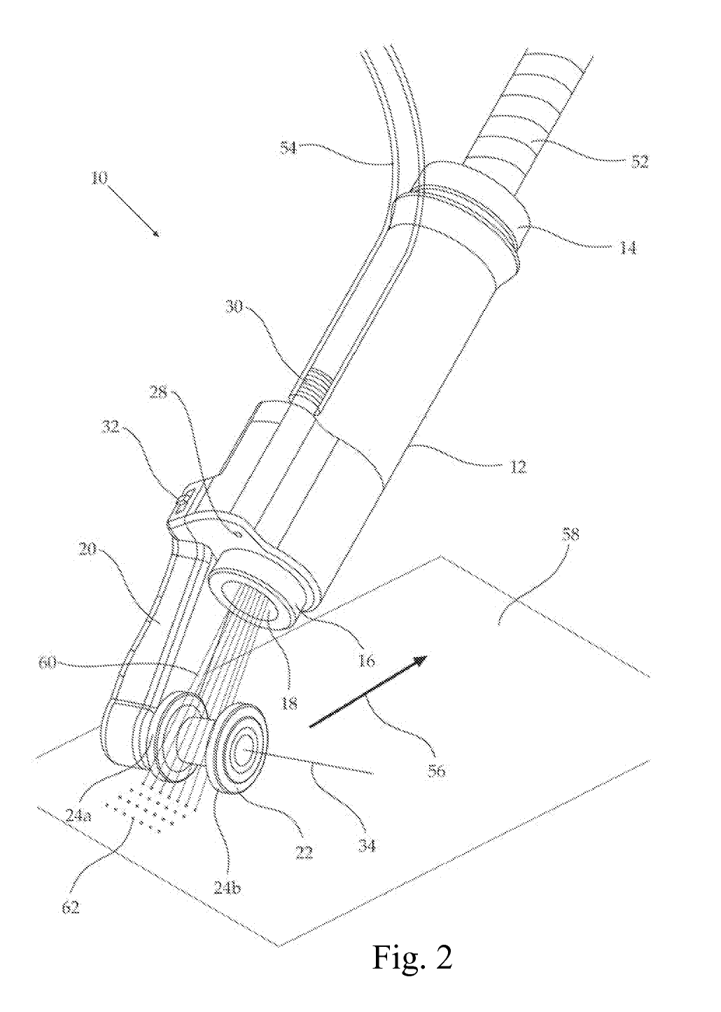

An embodiment of the method as described herein implemented using device 10 is described below with reference to FIG. 2.

An operator connects device 10 to a suitable external light source so that the light-output and the light-source trigger input of the external light source are functionally associated with light director 16 and trigger 26, respectively, by attaching a light-source adaptor 52 to device body 10 through light-source connector 14. The operator also attaches an air-hose 54 to nipple connector 30.

Using the standard control panel of the external light source, the operator adjusts the desired duration of each pulse of light (pulse width), the pulse intensity and other such parameters, in accordance with the disclosure herein.

The user selects a prescribed distance using distance-selector switch 32 in multiples of 0.59 mm, i.e., 0.59 mm, 1.18 mm, 1.77 mm.

The user activates a pressured gas source that provides a flow of gas (air, N₂) through air hose 54, nipple connector 30 and the air conduit to exit through air port 28. As a result, smoke and debris that is released during use of device 10 is blown away, preventing interference of the light beams by the smoke and debris generated by the effect of the light beams on the skin.

The user holds device body 10 in a hand, depresses the foot pedal and moves device 10 in a prescribed direction 56 perpendicular to axis 34 while maintaining contact of contact surface 24 with a skin surface 58 of a subject. As device 10 is moved, roller 22 rotates, axle 38 and consequently perforated disk 46 in bearing 44. The processor monitors the output signal from microphotosensor 50. Every time contact surface 24 is moved the prescribed distance selected by the user as measured by the extent of rotation of perforated disk 46, the processor sends a light-source triggering signal that triggers the external light source to produce a pulse of light. The pulse of light passes through device body 10 to pass through light director 16. The light exits light director aperture 18 of light director 16 towards skin surface 58 as a plurality of individual light beams 60 (in FIG. 2, seven light beams) that impinge on skin surface 58 substantially simultaneously. As described above, the fact that contact surface 24 is made up of two physically distinct surfaces 24a and 24b, assists in preventing device 10 from tilting, ensuring that beams 60 impinge on skin surface 58 at a desired predetermined angle.

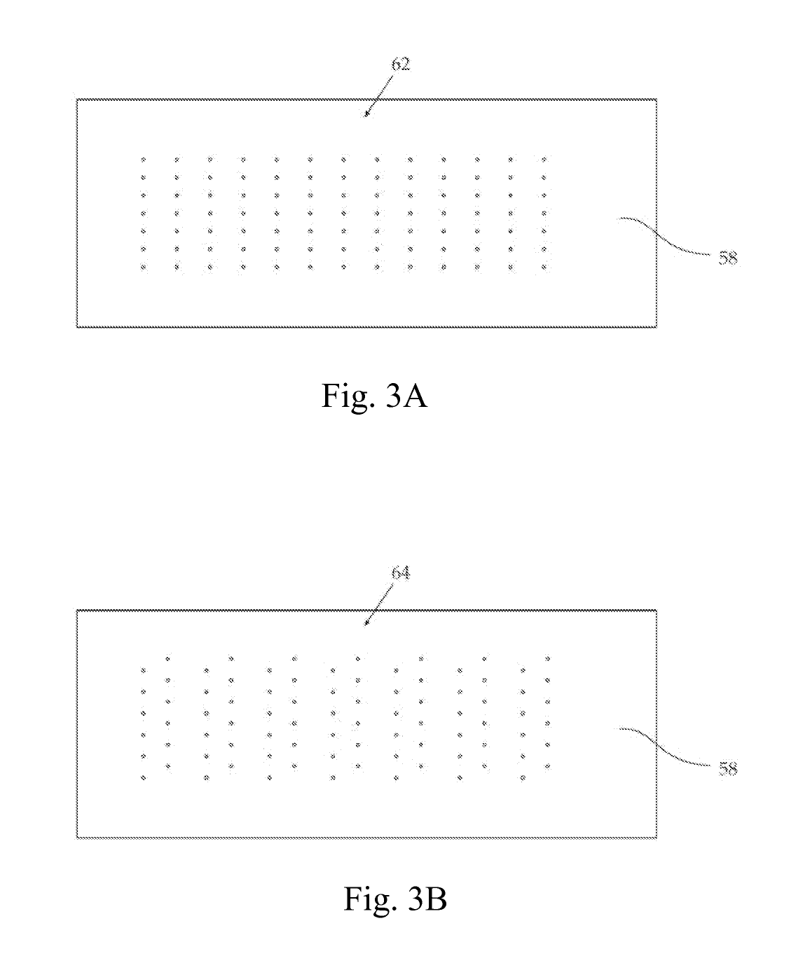

In the embodiment of a device described above, device 10, light director 16 is configured to direct a pulse of light produced by a functionally-associated light source at a skin surface 58 as a one-dimensional array of seven individual light beams 60 impinging on skin surface 58 and having a dimension of 1 cm perpendicular to prescribed direction 56, where at the skin surface each light beam 60 has a round cross-sectional shape of 0.03 mm² (a 0.2 mm diameter circle). A portion of a trail 62 of illuminated spots produced by such a device on a skin surface 58 is schematically depicted in FIG. 2 and FIG. 3A.

As discussed above, embodiments of a light director of a device as described herein are configured to produce light beams having different characteristics and different patterns of illuminated spots, for example: more or fewer than seven individual light beams; the light beams impinge on a skin surface within an area of skin having different dimensions (wider or narrower than the 1 cm wide pattern of device 10); a greater or lesser cross-sectional array than 0.03 mm²; a different shape than round; and/or in a different pattern. For example, in FIG. 3B, a portion of a trail 64 of illuminated spots produced by a device configured to produce twelve light beams that simultaneously impinge on a skin surface 58 arranged as two staggered one-dimensional arrays is schematically depicted.

In device 10, light director 16 is a user-replaceable diffraction beam splitter. When desired, a user can remove a given light director 16 from device body 10 (by unscrewing) and attaching a different light director 16, for instance, a diffraction beam splitter having different characteristics such as number of individual beams of light, shape of the beams of light and dimensions of the beams of light.

In device 10, spacer arm 20 is fixedly attached to the rest of device body 10 so that the distance of light director aperture 18 from roller 22 (and consequently from a skin surface 58 during use) is fixed. In some embodiments, the distance is user-adjustable, for example, a spacer arm is telescopic or moveably attached to the rest of a device body, allowing the distance to be user-adjustable.

In device 10, spacer arm 20 is fixedly attached to the rest of device body 10 so that the angle of light director aperture 18 relative to roller 22 is fixed, and consequently the incident angle of light beams 60 to a skin surface 58 during use is substantially constant. In some embodiments, the angle is user-adjustable, for example, a spacer arm is rotatably attached to the rest of a device body (e.g., through an axis parallel to axis 34), allowing a user to adjust the angle of the light director aperture relative to the roller and thus the incident angle of the light beams to the skin surface to be adjustable.

Device 10 described above is reversibly functionally associatable with an external light source. For use, an operator associates device 10 with a suitable available external light source and uses device 10 together with the external light source in accordance with the teachings herein. In some embodiments, a user has a number of devices, substantially similar to device 10, having different parameters, for example different characteristics relating to the light beams (e.g., number of light beams, light beam size, light beam pattern or length of spacer arm). For use, an operator selects one of the plurality of devices, functionally associates the selected device to the external light source, and uses the device together with the external light source in accordance with the teachings herein.

In some embodiments, a light source such as the external light source discussed above is considered a component of a device such as device 10, so that when the device is referred to, the light source is included as a component thereof.

As described above, during use of device 10, a pressured gas source is functionally associated through nipple connector 30 allowing smoke and debris produced during use of device 10 to be blown away. In some embodiments, a suction source is functionally associated with a device such as device 10 to draw smoke and debris away.

As described above, distance measurer 36 of device 10 described herein comprises a processor comprising an electronic microprocessor. In some related embodiments, a distance measurer is devoid of such a microprocessor, especially a distance measurer comprising a shaft encoder. In such embodiments, a signal indicative of a distance traveled in the prescribed direction is used directly as a light-source triggering signal. For example, when such a device is activated, the signal produced by a photosensor of a microphotosensor such as 50 upon detection of light produced by the light-emitting diode is used as a light-source triggering signal.

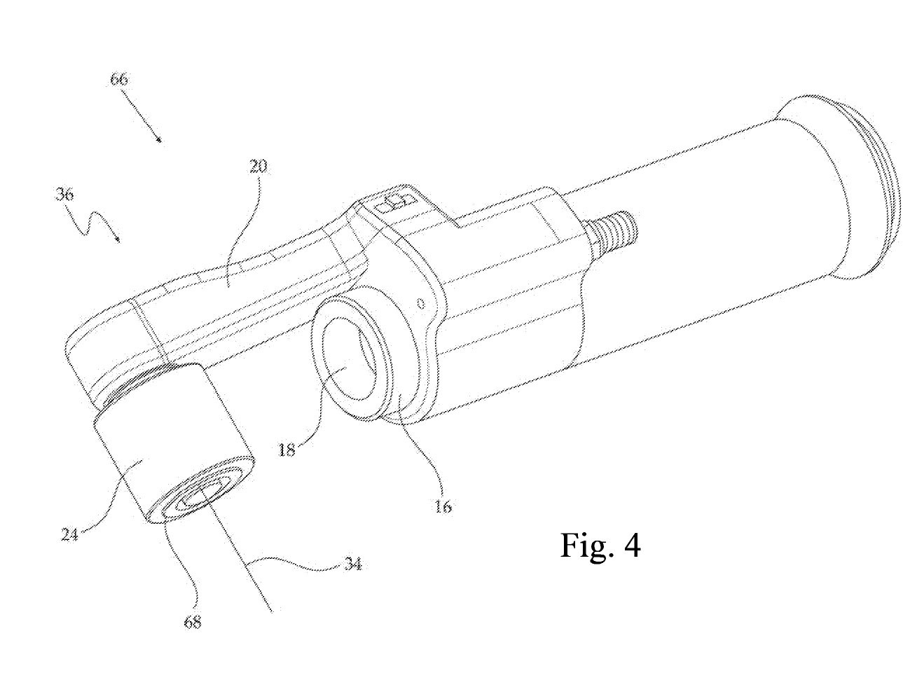

An embodiment of a dermatological treatment device as disclosed herein, device 66, is schematically depicted in FIG. 4. Device 66 is substantially similar to device 10 discussed above.

Distance measurer 36 of device 66 is substantially similar to distance measurer 36 of device 10 and comprises a mechanical rolling component bearing a contact surface that rolls along a skin surface during use to determine a distance traveled in a prescribed direction when in contact with a skin surface. In device 66, roller 68 is an 18-mm diameter cylinder with a width of 2.5 cm instead of a spool-shaped roller 22 of device 10. Contact surface 24 is the surface of roller 68 where, during use, substantially only a 2.5 cm wide strip of contact surface 24 perpendicular to the prescribed direction makes actual physical contact with a skin surface at any given time.

As roller 68 is cylindrical (and not spool-shaped), contact surface 24 flattens the area of skin on which the plurality of light beams directed by light director 16 impinge.

Light director 16 of device 66 is configured to direct beams of light past the edge of roller 68 and contact surface 24 and not between portions 24a and 24b of contact surface 24 as in device 10.

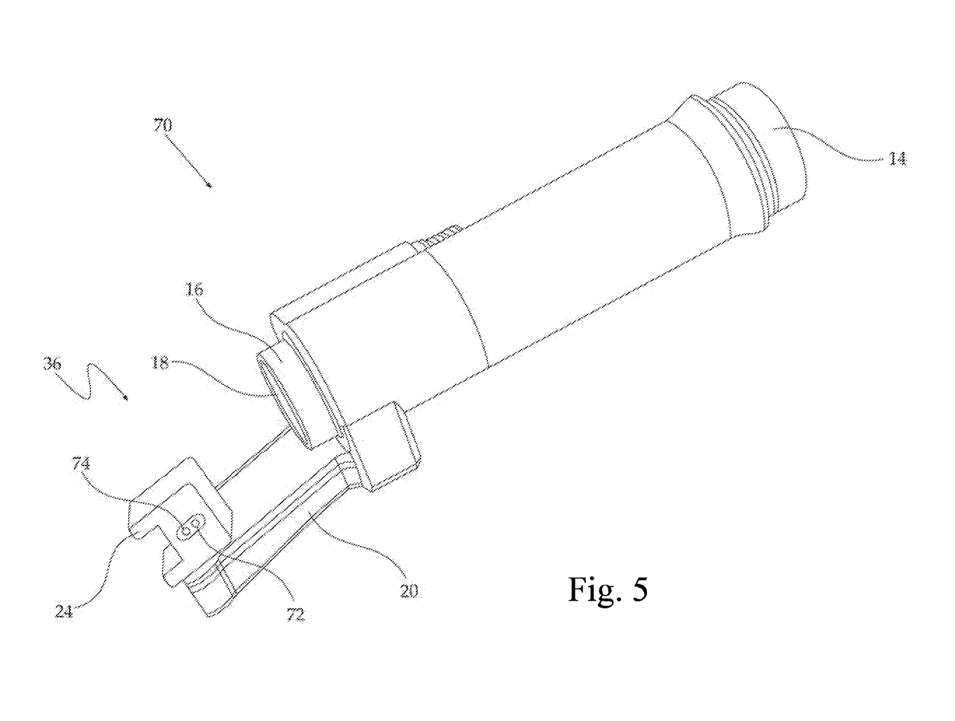

An embodiment of a dermatological treatment device as disclosed herein, device 70, is depicted in FIG. 5 in a perspective view from below to show components of distance measurer 36.

Device 70 is substantially similar to devices 10 and 66 discussed above.

In device 70, a distance measurer 36 does not comprise a mechanical rolling component. Instead, distance measurer 36 of device 70 functions in a manner substantially similar to that of an optical computer mouse, and is functionally associated with contact surface 24, a planar non-moving surface 2.5 cm wide (perpendicular to the prescribed direction) and 2.5 cm deep (parallel to the prescribed direction). Distance measurer 36 further comprises an optoelectronic sensor 72 and a dedicated distance-measurer illuminator 74 (an infrared laser diode as known in the art of laser mice for computer input) both embedded in contact surface 24 functionally associated with an image-processor (hidden from view in FIG. 5). Sensor 72 functions as a camera that periodically (1500 Hz) acquires images while the image processor compares succeeding images and translates changes in the images to distance. Distance measurer 36 is configured (as known in the art of computer mice, for example by determining the intensity of light from illuminator 74 that is detected by optoelectronic sensor 72) to determine whether or not there is contact between contact surface 24 and a surface (e.g., a skin surface). The trigger of device 70 is configured to avoid triggering the light source if there is no contact with a surface.

Due to the shape and dimensions, contact surface 24 is configured to flatten the surface of the area of skin on which the plurality of light beams directed by light director 16 impinge.

The dimensions and shape of contact surface 24 assist in maintaining a desired orientation of light director aperture 18 relative to a skin surface during use: as contact surface 24 is relatively wide and relatively deep, contact surface 24 helps prevent tilting (in a plane parallel to the prescribed direction and in a plane perpendicular to the prescribed direction) of light director aperture 18 relative to a skin surface.

Like device 10, device 70 includes a component configured to clearly delineate the area of a skin surface that is illuminated with the beams of light to a user. Specifically, contact surface 24 of device 70 includes two projections, and device 70 is configured so that the beams of light from light director aperture 18 are directed to pass between the two projections of contact surface 24 and thus fall inside a skin surface delineated by the two projections.

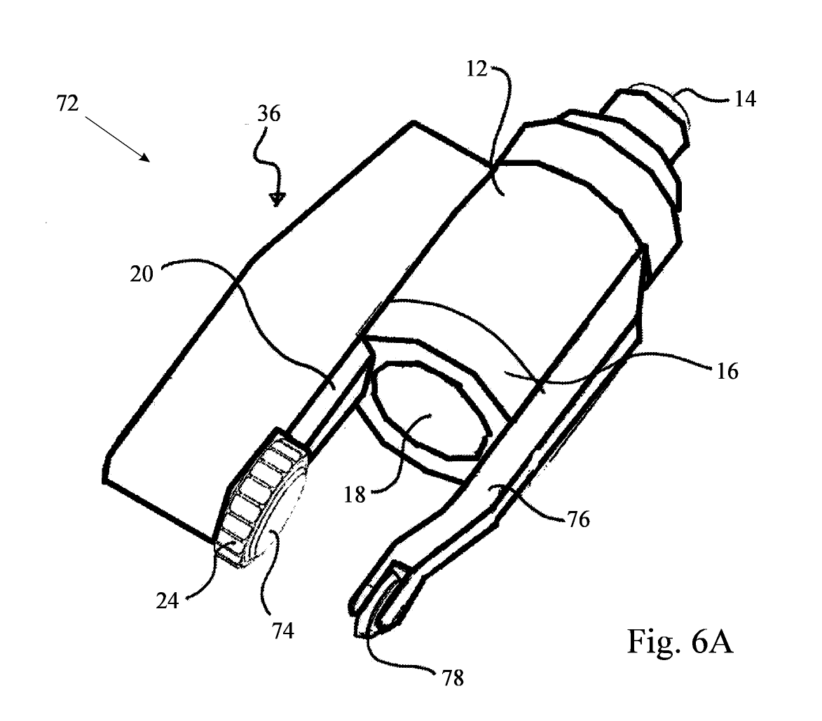

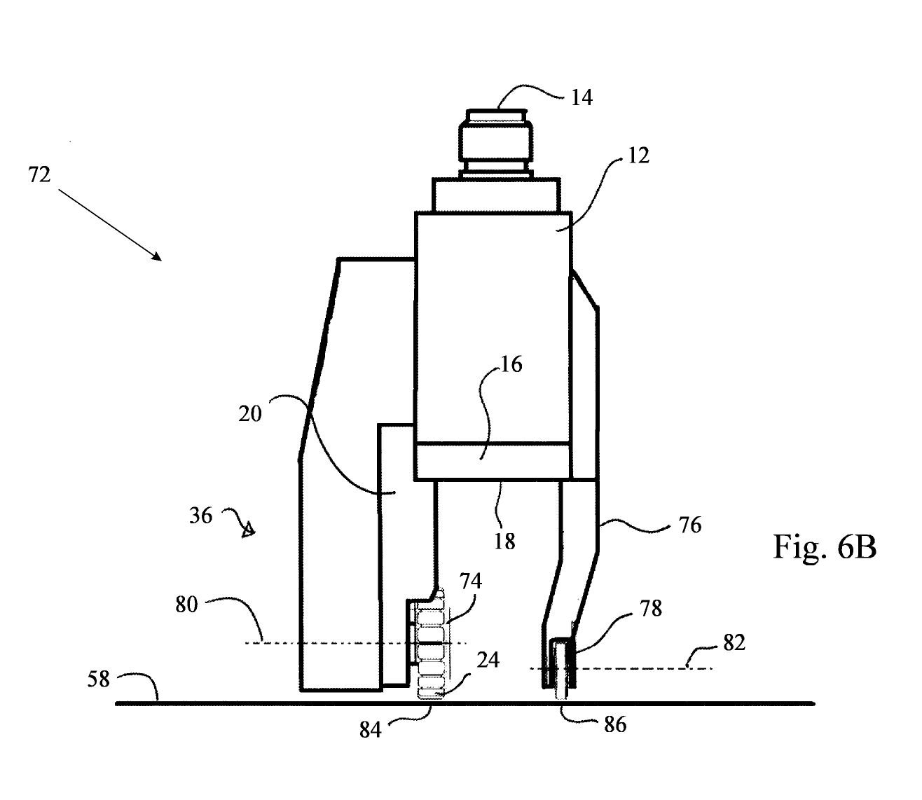

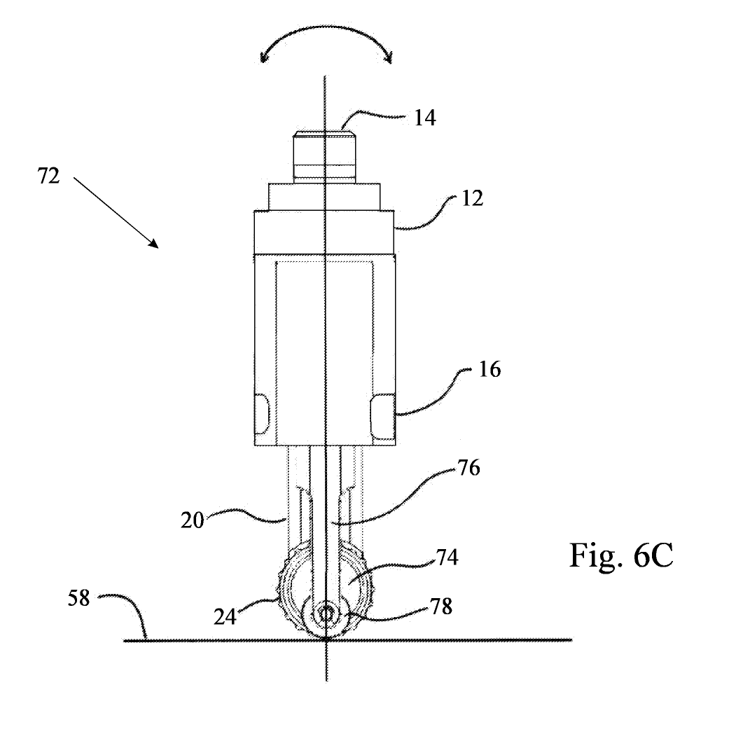

An additional embodiment of a dermatological treatment device as disclosed herein, device 72, is depicted in FIG. 6A (perspective view), FIG. 6B (front view) and FIG. 6 (C side view).

Device 72 comprises a device body 12 containing various components including a light-source connector 14, a user-replaceable light director 16 including a light-director aperture 18, a spacer arm 20 to which distal end is attached a 8 mm radius measuring wheel 74 bearing a contact surface 24 that constitutes a component of a distance measurer 36 of device 72, and a dummy spacer arm 76 to which distal end is attached a 4 mm radius dummy wheel 78. Device 72 is configured so that the view to where the light beams impinge on a skin surface is unobscured by device 72 and that device 72 does not come in physical contact with where the light beams impinge on the skin surface.

Measuring wheel 74 is configured to rotate exclusively around a rotation axis 80 and is functionally associated with a shaft encoder (substantially as described above with reference to device 10). Dummy wheel 78 is configured to rotate exclusively around a rotation axis 82 which is parallel to rotation axis 80. Device 72 and especially spacer arms 20 and 76, measuring wheel 74 and dummy wheel 78 are together configured so that when device body 12 is held perpendicularly to skin surface 58 (in FIGS. 6B and 6C), contact surface 24 of measuring wheel 74 makes frictional contact with skin surface 58 at a first location 84 on skin surface 58 and the rim of dummy wheel 78 make frictional contact at a second location 86 on skin surface 58 so that light director aperture 18 is maintained at a height of 5 cm above skin surface 58 when device body 12 is held perpendicularly to skin surface 58.

During use, a short-duration pulse of light for dermatological treatment produced by a light-source (not depicted) is directed to device body 12 through light-source connector 14 substantially as described above. The light passes through a beam splitter component of light director 16 that splits the light into a seven (in some related embodiments, nine) coplanar individual light beams, each beam having a 2° beam divergence. The seven individual light beams pass through an optical focussing lens component of light director 16 that focuses the individual beams to a focal plane parallel with axes 80 and 82 and 5 cm from light director aperture 18. Light director 16 and especially the focussing lens thereof is configured so that when device body 12 is held perpendicularly relative to skin surface 58 (in FIGS. 6B and 6C), the focal plane is coincident with skin surface 58, and the seven individual light beams impinge on skin surface 58 perpendicularly as seven illuminated points (200 micrometer diameter) spaced from one another (1 mm center-to-center) along a line between first location 84 and second location 86 of skin surface 58. Accordingly, device 72 is configured to be used so that the focal plane of the individual light beams coincides with skin surface so that the light beams preferably impinge on skin surface 58 substantially perpendicularly.

In device 72, measuring wheel 74 and dummy wheel 78 together constitute a flanking component, flanking a target area of skin surface 58. Specifically, when device 72 is held so that the individual light beams impinge on a skin surface at a preferred angle (substantially perpendicularly), the light beams impinge on a target area of skin surface between locations 84 and 86.

A use of device 72 is analogous to the described above with reference to device 10. A user holds device body 12 generally perpendicularly to a skin surface 58 so that measuring wheel 84 and dummy wheel 86 make frictional contact with skin surface 58. While maintaining frictional contact of wheels 84 and 86 with skin surface 58, the user moves device body 12 in a prescribed direction that is perpendicular to axes 80 and 82. Such movement leads to rotation of measuring wheel 84, which is a measure of the distance traveled by contact surface 24 along skin surface 58. As described above, rotation of measuring wheel 84 by a preset angle that corresponds to a prescribed distance generates a triggering signal that triggers the light source, producing a line of seven illuminated points between first location 84 and second location 86.

Since contact surface 24 and light director aperture 18 are mutually physically associated so that the distance therebetween is fixed, during use of device 72 light director aperture 18 is maintained at a substantially constant height of 5 cm from skin surface 58 when device body 12 is held so that the light beams impinge on skin surface 58 at the preferred angle (perpendicular to skin surface 58). As contact with skin surface 58 is only through first location 84 and second location 86, device 72 is configured so that during use device body 12 can undergo pitching, that is to say rotated in a plane including the prescribed direction and substantially perpendicularly to skin surface 58 while maintaining contact of contact surface 24 with skin surface 58. A user can choose to stop moving device body 12 to examine portions of skin surface 58 already illuminated or to be illuminated by pitching device body 12 without losing contact of contact surface 24 with skin surface 58. During use, a user may also pitch device body 12 to some extent (typically not more than ±20°) while moving device 72 for comfort or to get a better view of an illuminated area. Such pitching is around axis 80 of measuring wheel 74. Due to the difference in radius between measuring wheel 74 and dummy wheel 78, device body 72 rotates sideways (listing around an axis parallel to the designated direction) but such sideways rotation has only an insubstantial effect on where the light beams impinge on skin surface 58. Additionally, due to the relatively small radius of measuring wheel 74 the path length the light beams travel from aperture 18 to skin surface 58 increases only slightly, leading to only insubstantial defocusing of the light beams.

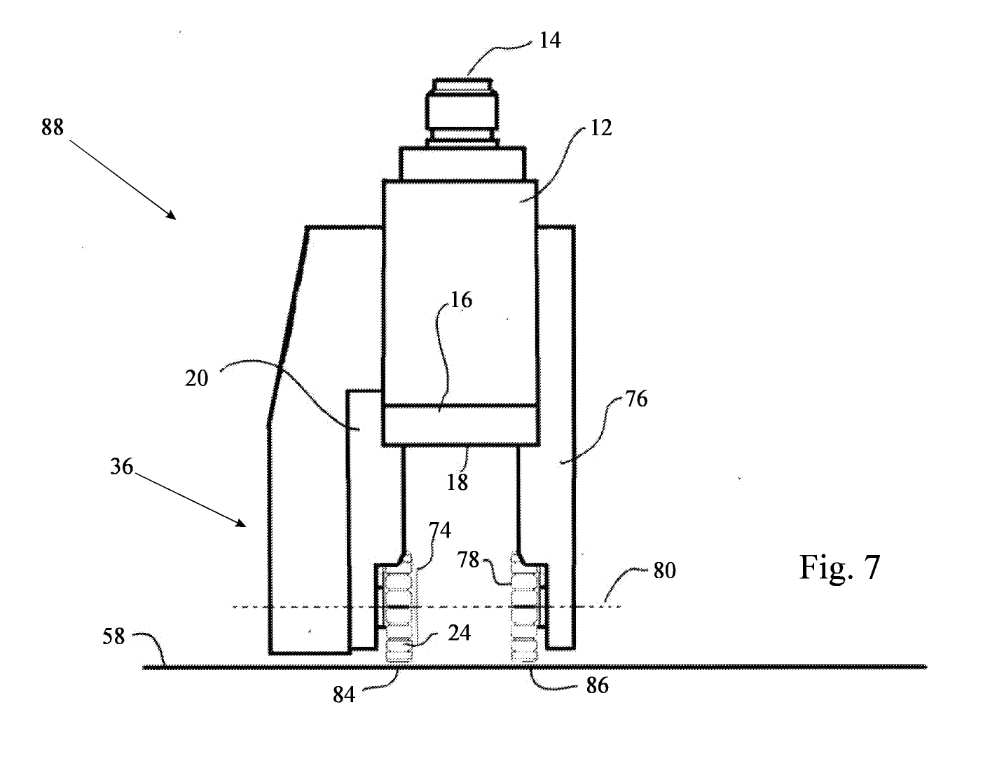

An additional embodiment of a dermatological treatment device as disclosed herein, device 88, is depicted in FIG. 7 in front view. Device 88 is substantially similar to device 72 described above, except that a dummy wheel 78 is substantially identical in size and shape to a measuring wheel 74 and the length of dummy spacer arm 76 is shortened. Accordingly an axis of rotation 80 of wheels 74 and 78, is the same, and any pitching of device body 72 does not lead to listing.

Pitching of device bodies 12 of devices 72 and 88 is around a rotation axis 80 of a measuring wheel 74. Accordingly, for a given pitch angle the increase in path length of light beams from an aperture 18 to a skin surface 58 is dependent on the radius of a respective measuring wheel 74. Accordingly, in some embodiments it is preferred that a measuring wheel be relatively small, typically not more than about 15 mm, not more than about 10 mm and in some embodiments even not more than about 5 mm.

As discussed above, devices 72 and 88 are configured for preferably directing a plurality of individual light beams substantially perpendicularly to a skin surface 58 by focusing the light beams in a focal plane that is coincident with a skin surface 58 when a device body 12 is perpendicular to the skin surface and by focussing the beams at a plurality of illuminated points along a line lying in the focal plane in the same line as the contacting locations 84 and 86. That said, in some embodiments, a device is configured for preferably directing a plurality of individual light beams to impinge on a skin surface at a preferred angle between about 5° and about 60°, in some embodiments of between about 25° and about 50°, from perpendicular to a skin surface. In some such embodiments, such configuration includes focussing the beams in a focal plane that intersects the skin surface when the beams impinge on the skin surface at the preferred angle. Additionally or alternatively, in some such embodiments such configuration includes directing the beams to impinge in the skin surface at a target area defined by a flanking component. Some such embodiments provide a clear view of an illuminated area of a skin surface during use.

As discussed above, devices 72 and 88 include a measuring wheel 74 which rim constitutes a contact surface 24. Additionally, devices 72 and 88 include a dummy wheel 76 which, inter alia, makes the device easier to use, allows pitching but assists in preventing listing of a device body 12 and assists a user in moving a contact surface 24 in the designated direction.

The light directors 16 of devices 72 and 88 are configured so that each one of the individual light beam is convergent and the focal points of all the light beams are coplanar. In some related embodiments, a light director is configured so that each one of the individual light beams is substantially not convergent.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

Section headings are used herein to ease understanding of the specification and should not be construed as necessarily limiting.

1. A dermatological treatment device comprising:
a) a trigger functionally associated with a distance measurer functionally associated with a contact surface,
said distance measurer configured to determine a distance traveled by said contact surface along a skin surface in a prescribed direction when in contact with said skin surface,
said trigger functionally associatable with a light source configured to produce a short-duration pulse of light when triggered, and
said trigger configured to repeatedly trigger a functionally-associated light source as said contact surface is moved along said skin surface when said distance traveled is a prescribed distance in said prescribed direction; and
b) functionally associatable with a said light source associated with said trigger, a light director including a light director aperture, wherein said light director is configured to direct a produced said short-duration pulse of light from said light director aperture at a said skin surface as a plurality of individual light beams.

2. The device of claim 1, further comprising a light source configured to produce a short-duration pulse of light when triggered, functionally associated with said trigger and with said light director.

3. (canceled)

4. The device of claim 1, configured so that the view to where said light beams impinge on said skin surface is unobscured by the device.

5. The device of claim 1, configured so that during use the device does not come in physical contact with where said light beams impinge on said skin surface.

6. The device of claim 1, configured so that during use said light director aperture is maintained at a substantially constant height from a skin surface.

7. The device of claim 6, wherein said substantially constant height is at least about 2 cm.

8. The device of claim 1, wherein said contact surface of said distance measurer and said light director aperture of said light director are mutually physically associated through a device body, so that a distance between said light director aperture and said contact surface is defined, so that during use of the device said light director aperture is maintained at a substantially constant height from a skin surface.

9. The device of claim 8, wherein said distance between said light director aperture and said distance measurer is user adjustable.

10-24. (canceled)

25. The device of claim 1, wherein said prescribed distance is adjustable by a user.

26-34. (canceled)

35. The device of claim 1, configured to be hand-operable.

36. (canceled)

37. The device of claim 1, wherein said distance measurer comprises a mechanical rolling component with a rolling surface constituting at least a part of said contact surface, said rolling component configured to exclusively rotate around an axis that is substantially perpendicular to said prescribed direction when said contact surface contacts a skin surface.

38. The device of claim 37, devoid of a digital processor for determining said distance moved by said contact surface along a skin surface.

39. (canceled)

40. The device of claim 1, wherein said contact surface is configured to assist in maintaining a desired orientation of said light director aperture relative to a skin surface during use.

41. The device of claim 1, wherein said contact surface of said distance measurer and said light director aperture of said light director are mutually physically associated through a device body, so that during use a distance between said light director aperture and said contact surface is fixed, so that the device is configured to allow rotation of the device body in a plane including said prescribed direction and substantially perpendicularly to a said skin surface while maintaining said contact surface in contact with a skin surface.

42-44. (canceled)

45. The device of claim 1, wherein said contact surface of said distance measurer and said light director aperture of said light director are mutually physically associated through a device body, so that during use a distance between said light director aperture and said contact surface is fixed, further comprising a flanking component configured to flank a target area of said skin surface; and
configured so that said individual light beams impinge on a said target area of said skin surface when impinging on said skin surface at a preferred angle.

46. The device of claim 45, wherein said flanking component comprises two distinct spaced-apart surfaces configured to contact a said skin surface at two respective distinct spaced-apart locations during use of the device.

47. The device of claim 46, configured so that said individual light beams impinge proximal to a line drawn between said two spaced-apart locations on a said skin surface when impinging on said skin surface at a preferred angle.

48-49. (canceled)

50. The device of claim 1, wherein said contact surface constitutes a portion of said flanking component.

51. The device of claim 50, wherein said distance measurer comprises a mechanical rolling component constituting a portion of said flanking component, said mechanical rolling component having a rolling surface constituting at least a part of said contact surface, said rolling component configured to rotate exclusively around an axis that is substantially perpendicular to said prescribed direction when said contact surface contacts a skin surface.

52. A dermatological treatment device comprising:
a light source configured to produce a short-duration pulse of light when triggered mounted on a device body;
said light source functionally associated with a light director including a light director aperture, wherein said light director is configured to direct a produced said pulse of light from said light director aperture as a plurality of individual light beams, so that the pulse of light is focused at a plurality of illuminated points that are spaced from one another along a line lying in focal plane at a predetermined distance from said light director aperture; and
functionally associated with said device body, a distance measurer configured to trigger said light source each time said device body is displaced along the focal plane by a prescribed distance in a prescribed direction transverse to the line of the illuminated points;
wherein said distance measurer includes:
a measuring wheel mounted on said device body for rotation about an axis generally parallel to the line of points, and
a detector functionally associated with said measuring wheel for generating a triggering signal for triggering said light source in response to each detection of a rotation of said wheel through a preset angle,
wherein said measuring wheel is operative to make frictional contact with said focal plane; and
wherein when said device body is oriented so that said light beams impinge on said focal plane at a preferred angle, said measuring wheel is operative to make frictional contact with said focal plane at a point lying on substantially the same line as, and laterally offset from, said illuminated points.

53. A method of dermatological treatment of an area of skin of a subject, comprising:
a) providing a device comprising a light director with a light director aperture and a trigger functionally associated with a light source, said light director configured to direct a short-duration pulse of light produced by said light source through said light director aperture at a skin surface when said light source is triggered;
b) moving said light director aperture in a prescribed direction relative to said skin surface; and
c) every time said light-director aperture travels a prescribed distance relative to said skin surface:
triggering said light source to produce a said short-duration pulse of light, and
directing said produced short-duration pulse of light at an area of said skin surface as a plurality of individual light beams
thereby causing a beneficial effect as a result of said pulse of light impinging on said skin surface
wherein said trigger is functionally associated with a contact surface,
wherein said moving is while maintaining contact of said contact surface with said skin surface; and
wherein said prescribed distance is a distance travelled by said contact surface along said skin surface in said prescribed direction.

54. The method of claim 53, wherein said plurality of individual light beams impinge on the skin surface substantially simultaneously.

55. The method of claim 53, wherein during said moving, the view to where said light beams impinge on said skin surface remains unobscured by said device.

56. The method of claim 53, wherein during said moving, said device does not come in physical contact with where said light beams impinge on said skin surface.

57. The method of claim 53, where said device is configured so that said light director aperture is maintained at a substantially constant height from said skin surface.

58. The method of claim 57, wherein said substantially constant height is at least about 2 cm.

59. The method of claim 53, wherein said light director aperture and said contact surface are mutually physically associated through a device body, so that a distance and relative orientation between said light director aperture and said contact surface are fixable during use, so that said pulse of light impinges on said skin surface at a prescribed location and incident angle relative to said contact surface.

60. The method of claim 59, wherein said incident angle is between about 5° and about 60° from perpendicular to said skin surface.

61. The method of claim 53, wherein said moving of said light director aperture is performed manually.

62. (canceled)
